(19) United States
(12) Patent Application Publication
(10) Pub. No.: US 2003/0211495 A1
(43) Pub. Date: Nov. 13, 2003

(54) Reverse n-hybrid screening method

(76) Inventors: Richard Hopkins, North Perth (AU); Ilya Serebriiskii, Philadelphia, PA (US); Paul Michael Watt, Mount Claremont (AU); Erica Golemis, Oreland, PA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/221,276

(22) PCT Filed: Mar. 8, 2001
(86) PCT No.: PCT/US01/07669

(30) Foreign Application Priority Data
Mar. 8, 2000 (AU) PQ6131
Mar. 23, 2000 (AU) PQ6437
Apr. 11, 2000 (AU) PQ6830
Nov. 6, 2000 (AU) PR1256

(51) Int. Cl. C12Q 1/68; G01N 33/53; G01N 33/567; C12N 15/85

(52) U.S. Cl. 435/6; 435/7.2; 435/325; 435/455

(57) ABSTRACT

The present invention relates provides an improved reverse n-hybrid screening method for identifying antagonists or inhibitors of biological interactions, wherein multiple reporter genes are employed to distinguish antagonists or inhibitors of one interaction from those antagonists or inhibitors of other interactions, particularly where each of said interactions involves one or more common interacting partners. A further aspect of the invention provides novel expression vectors for performing the inventive method, particularly in high throughput screens.

### FIELD OF THE INVENTION

The present invention relates generally to an improved reverse n-hybrid screening method for identifying antagonists or inhibitors of biological interactions, wherein multiple reporter genes are employed to distinguish antagonists or inhibitors of one interaction from those antagonists or inhibitors of other interactions, such as, for example, wherein each of said interactions involves one or more identical interacting partners. The present invention provides the means by which a wide range of peptide-based therapeutic, prophylactic and diagnostic reagents may be developed.

### GENERAL

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any two or more of said steps or features.

Throughout this specification, unless the context requires otherwise the word “comprise”, and variations such as “comprises” and “comprising”, will be understood to imply the inclusion of a stated integer or step or group of integers or steps but not the exclusion of any other integer or step or group of integers or steps. The present invention is not to be limited in scope by the specific embodiments described herein, which are intended for the purposes of exemplification only. Functionally-equivalent products, compositions and methods are clearly within the scope of the invention, as described herein.

Bibliographic details of the publications referred to by author in this specification are collected at the end of the description. Reference herein to prior art, including any one or more prior art documents, is not to be taken as an acknowledgment, or suggestion, that said prior art is common general knowledge in Australia or forms a part of the common general knowledge in Australia.

As used herein, the term “derived from” shall be taken to indicate that a particular integer or group of integers has originated from the species specified, but has not necessarily been obtained directly from the specified source.

This specification contains nucleotide sequence information prepared using the program Patent in Version 3.0, presented herein after the claims. Each nucleotide sequence is identified in the sequence listing by the numeric indicator <210>followed by the sequence identifier [e.g. <210>1, <210>2, etc]. The length, type of sequence [DNA, protein (PRT), etc] and source organism for each nucleotide sequence are indicated by information provided in the numeric indicator fields <211>, <212>and <213>, respectively. Nucleotide sequences referred to in the specification are defined by the term “SEQ ID NO:”, followed by the sequence identifier [e.g. SEQ ID NO: 1 refers to the sequence in the sequence listing designated as <400>1].

The designation of nucleotide residues referred to herein are those recommended by the IUPAC-IUB Biochemical Nomenclature Commission, wherein A represents Adenine, C represents Cytosine, G represents Guanine, T represents thymidine, Y represents a pyrimidine residue, R represents a purine residue, M represents Adenine or Cytosine, K represents Guanine or Thymidine, S represents Guanine or Cytosine, W represents Adenine or Thymidine, H represents a nucleotide other than Guanine, B represents a nucleotide other than Adenine, V represents a nucleotide other than Thymidine, D represents a nucleotide other than Cytosine and N represents any nucleotide residue.

### BACKGROUND OF THE INVENTION

Biological interactions, such as, for example, a protein-protein interaction or protein-nucleic interaction, are involved in a wide variety of processes occurring in living cells. For example, agonism or antagonism of a receptor by a specific ligand, such as, for example, a drug, hormone, second messenger molecule, etc., may effect one or more of a variety of biological processes, including gene expression, cellular differentiation, growth, enzyme activity, metabolite flow, or metabolite partitioning between cellular compartments. Protein-protein interactions, DNA-protein interactions, and RNA-protein interactions, are well known for their effects in regulating such biological processes in both prokaryotic and eukaryotic cells, in addition to being critical for DNA replication, and, in the case of RNA viruses, RNA replication.

Undesirable or inappropriate gene expression and/or cellular differentiation, cellular growth and metabolism may be attributable, at least in many cases, to biological interactions involving the binding and/or activity of proteinaceous molecules, such as, for example, transcription factors, peptide hormones, receptor molecules, or enzymes. In one example, several genes activated by chromosomal translocations in lymphoid malignancies code for transcription factors, for example MYC, LYL-1 and SCL, which appear to function via protein-protein interactions. In normal cells, these proteins are in an appropriate equilibrium with their interaction partners which is disturbed as a consequence of oncogene activation and is thought to result in transcription of target genes normally expressed in other cells or lineages. These transcription factors may also substitute for, or antagonize, the function of closely related endogenous proteins to perturb gene expression essential for normal growth control.

Peptides present potential therapeutic and prophylactic agents for many human and animal diseases, biochemical disorders and adverse drug effects, because they can interact with other molecules highly specifically. For example, mimetic peptides have been reported to inhibit protein interactions and/or enzymatic functions. More specific examples include a nonapeptide derived from the ribonucleotide reductase of herpes simplex virus linked to an enterotoxin subunit for delivery into cells via its receptor. The peptide conjugate is found to inhibit herpes simplex type I replication in quiescent Vero cells (Marcello et al. 1994). Using detailed knowledge of the PCNA-interaction domain of p21^{WAF1}, a peptide was designed which effectively blocked the interaction. This 20-mer peptide bound with sufficient affinity to block SV40 replication (Warbrick et al. 1996). A 20-mer peptide sequence derived from p16 was found to interact with cdk4 and cdk6 and inhibited pRB phosphorylation and cell cycle progression (Fahraeus 1996). Peptides have even been shown to function as inhibitors in animal models. For examples, a peptide targeting the ICE protease was shown to be a potent protective inhibitor against liver apoptosis induced by TNF-α in the mouse (Rouquet et al. 1996). RNA-protein interactions, such as, for example, the TAT/TAR interaction of HIV, or the interaction of RNA with the protein subunits of telomerase, present attractive targets for therapy.

Conventional forward n-hybrid screens have proven useful for identifying proteins that specifically interact with a target protein of interest in a cell. In such assays, the protein of interest is generally expressed as a fusion protein with the DNA binding domain (DBD) of a transcription factor, and a transcription activator domain (AD) is expressed separately as a fusion with each member of an array of peptides. When the appropriate association between binding partners occurs in the cell, a functional transcription factor is reconstituted, and expression of a reporter gene placed under the control of the reconstituted transcription factor occurs. The cell expressing the appropriate binding partner can then be isolated, facilitating identification of the binding partner. Significantly, the selection system used is a positive selection, wherein expression of the reporter gene is enhanced, by the interaction between the binding partners.

Conversely, reverse n-hybrid screening technologies provide a direct means of isolating antagonists or “peptide inhibitors” of a given RNA-protein, DNA-protein, or protein-protein interaction. Such screens differ from canonical forward n-hybrid screens, by providing a selection against the interaction. This is achieved, for example, by utilizing a counter selectable reporter gene that encodes a lethal product when expressed in the cell, or alternatively, encodes an enzyme that converts a non-toxic substrate to a toxic product. Counter selectable reporter genes suitable for this purposes include, for example, the yeast URA3 structural gene which is lethal to yeast cells when expressed in the presence of 5-fluororotic acid (5-FOA); the yeast CYH2 gene which is lethal when expressed in the presence of the drug cycloheximide; and the yeast LYS2 gene which is lethal in the presence of the drug α-aminoadipate (α-AA). Reverse n-hybrid screens employing such counter selectable reporter genes are described in detail in International Patent Application No. PCT/AU99/00018 (WO 99/35282), which particularly describes a high-throughput reverse n-hybrid screening method for screening random peptide (i.e. aptamer) libraries in vivo.

The utility of reverse two hybrid approaches for high-throughput screening of small molecule drug libraries has also been described recently by Huang and Schreiber (1997) and by Young et al.(1998), albeit not with respect to the screening of aptamer libraries.

Many distinct biological interactions in complex organisms employ overlapping subsets of interacting partners, or partners that are organized into large protein families that are highly conserved at the amino acid sequence level, particularly in regions that are required for protein binding activity or nucleic acid binding activity. For example, the GTPases Ras and Krev-1 are 56% identical and are known to interact with an overlapping set of protein partners, albeit at different affinities, namely, Raf to which Ras preferentially binds, Krit-1 to which Krev-1 preferentially binds, and the RaI guanine dissociation stimulator protein (RaIGDS), to which both proteins bind (Serebriskii et a. 1999). Similarly, the transcription factor SCL, which is expressed in malignant lymphoid cells, interacts with LMO1, LMO2, DRG, mSin3A, and E47 proteins (Mahajan et al, 1996).

Despite the utility of reverse n-hybrid screens for identifying potentially useful therapeutic molecules that antagonize a simple interaction in a cell, such screens may not be capable of distinguishing between multiple interactions in the cell that involve binding partners having the same or closely related partner-binding domains, such as those described supra. Known reverse two hybrid or reverse three hybrid screens do not provide a means for preferentially selecting for one interaction between two or more binding partners over another protein-protein interaction involving one or more of the same binding partners, or closely related binding partners. Accordingly, existing technologies have the disadvantage of detecting false positives in the screening process, necessitating additional time and effort to resolve true binding partners.

Moreover, existing reverse two hybrid screens and reverse three hybrid screens are not ideally suited to high through-put applications due to a lack of suitable shuttle vectors or host cell strains.

### SUMMARY OF THE INVENTION

In work leading up to the present invention, the inventors sought to develop reverse n-hybrid screens having enhanced specificity in their detection of interacting binding partners compared to conventional reverse n-hybrid screens, and more particularly, having the capacity to distinguish between interactions that involve one or more common binding partners.

As used herein, the term “interaction” shall be taken to refer to a direct or indirect physical association between two or more molecules or “partners”, wherein said association is involved in a cellular process or alternatively, is required for said cellular process to occur. The “association” may involve the formation of an induced magnetic field or paramagnetic field, covalent bond formation such as, for example, a disulfide bridge formation between polypeptide molecules, an ionic interaction such as, for example, occur in an ionic lattice, a hydrogen bond or alternatively, a van der Waals interaction such as, for example, a dipole-dipole interaction, dipole-induced-dipole interaction, induced-dipole-induced-dipole interaction or a repulsive interaction or any combination of the above forces of attraction.

For example, in a reverse two hybrid assay, the interaction between the proteinaceous binding partners may involve their direct physical association (i.e. with no intervening molecule). Similarly, in a reverse three hybrid assay, the interaction between some of the binding partners, particularly the interaction between the two baits, and the interaction between one of the baits and the prey, is a direct physical association. However, in a three hybrid assay, the constant bait and the prey are clearly in indirect association with each other, because the non-constant bait (e.g. an RNA-bait in the case of an RNA/protein interaction) forms a “bridge between these partners. Accordingly, for the purposes of the present invention, the term “interaction” clearly includes within its scope such direct and indirect associations between the various binding partners, and, as a consequence, does not omit any unstated binding partners.

As used herein, the term “n-hybrid” refers to the number of binding partners, other than the peptide inhibitor, that are involved in an interaction that is assayed. Accordingly, there are two binding partners in a conventional reverse two hybrid screen and three binding partners in a conventional reverse three hybrid screen, and so on. Accordingly, one aspect of the present invention provides a method of identifying a peptide that partially or completely inhibits a target interaction between two or more binding partners in a host cell but does not inhibit a non-target interaction between some but not all of said binding partners, said method comprising:

(i) expressing in a cellular host: (a) the binding partners of said target interaction such that they operably control the expression of one or more reporter genes in said cellular host, wherein said expression is partially or completely inhibited by disruption of said target interaction; (b) the binding partners of said non-target interaction such that they operably control the expression of one or more reporter genes in said cellular host, wherein said expression is partially or completely inhibited by disruption of said non-target interaction and wherein said reporter gene is distinct from the reporter gene(s) expressed under control of the target interaction; and (c) a candidate peptide;

(ii) growing the cellular host under conditions sufficient to distinguish the expression of each reporter gene(s) at (a) from expression of the reporter gene(s) at (b); and

(iii) detecting those host cells wherein expression of the reporter gene(s) operably under control of the target interaction is(are) partially or completely inhibited and expression of the reporter gene(s) operably under control of the non-target interaction is(are) not inhibited, said detected cells expressing a peptide that partially or completely inhibits the target interaction.

This aspect of the invention clearly includes within its scope both reverse two hybrid and reverse three hybrid assays, or any reverse n-hybrid assay, by virtue of the term “interaction” as defined herein above. As will be known to those skilled in the art, the only difference between the reverse two hybrid format, and higher order formats, is the addition of further binding partners that may alter the relative physical associations between each of the binding partners, however the general principal for all reverse n-hybrid assay formats is the same.

The host cell may be any cell capable of supporting the expression of exogenous DNA, such as, for example, a bacterial cell, insect cell, yeast cell, mammalian cell or plant cell. Preferably, the cell is a bacterial cell, mammalian 35 cell, or a yeast cell preferably, a yeast cell will have the genotype MATα, ura3, trp1, his3, cyh2^{R}, lexAop-URA3,/exAop-CYHZ ade2. Such a yeast strain may be constructed using spontaneous cycloheximide-resistant derivatives of YPH252 (Sikorski et al., 1989) or EGY40 (Golemis and Brent, 1992; Gyuris et al. 1993).

Alternatively, the yeast strain can be PRT 473, a cycloheximide resistant derivative of strain SKY 473, of the genotype (MAT-α, cyh2^{R} his3, trp1, lexAop-LEU2, lexAop-CYH2ade2::ZEO^{R}, lexAop-URA3his5::G418^{R}, clop-LYS).

By “target interaction” is meant the interaction in the cellular host against which a specific antagonist or peptide inhibitor is sought.

A “non-target interaction” means an interaction that involves at least one common binding partner as defined herein above with the target interaction, however against which a specific antagonist is not being sought. Accordingly, it is an object of the present invention to exclude those peptides that antagonize both the target interaction and the non-target interaction in the cellular host, and to preferentially select those peptides that antagonize only the target interaction in the cellular host, or that antagonize the target interaction at a higher affinity than the antagonism of the non-target interaction.

The term “partial or complete inhibition” means that expression of the reporter gene under the control of the target interaction is reduced to a level that does not interfere with the capability to distinguish between the target interaction and the non-target interaction as defined herein above.

As used herein, the term “binding partner” shall be taken to mean protein or nucleic acid that binds to another protein or nucleic acid in a cell, including a complete protein or gene, or the dimerization region of a protein, or a cis-acting nucleotide sequence that binds to nucleic acid or protein. As will be known to those skilled in the art, at least two binding partners are involved in any biological interaction in a cell. The term “dimerization region” means that part of a protein that is in physical relation with a binding partner. The term “cis-acting nucleotide sequence” shall be taken to mean a nucleotide sequence that regulates gene expression by virtue of the binding of a repressor protein, or activator protein thereto, including the minimum nucleotide sequence that has the ability to bind said repressor protein or said activator protein.

Accordingly, it shall be clear to the skilled person that any reference herein to a common binding partner includes a reference to homologous proteins or genes that share one or more dimerization regions or cis-acting nucleotide sequences, respectively, with a known binding partner that is involved in an interaction against which the reverse hybrid screen of the invention is directed (i.e. the target interaction).

Thus, in cases of large protein families, or large multigene families, it is an object of the present invention to select a peptide that partially or completely inhibits a target interaction between two or more binding partners in a host cell but does not inhibit a non-target interaction between some but not all of said binding partners, such as, for example, any homologues of said binding partners that share common dimerization regions or cis-acting sequences, or alternatively, identical binding partners that themselves interact with a number of different binding partners.

In a particularly preferred embodiment of the invention, one or more of the binding partners of the non-target interaction is the same as a binding partner of the target interaction.

The invention is not to be limited by the nature of the target interaction being assayed, or the nature of the non-target interaction, because all formats are readily determined by those skilled in the art without undue experimentation based upon the disclosure contained herein. Preferably, one or more of the binding partners is comprises amino acids, such as, for example, a peptide, polypeptide, protein or enzyme molecule, or alternatively, comprises nucleic acid, such as DNA, RNA, or a DNA/RNA hybrid. More preferably, the target and/or non-target interactions are/is a protein/protein interaction or protein/nucleic acid interaction.

Preferably, the number of binding partners involved in each interaction is at least two. In a particularly preferred embodiment, present invention provides an improved reverse two hybrid assay, wherein:

(i) the binding partners of the target interaction consist of two fusion proteins, wherein (a) one fusion protein comprises the DNA binding domain of a transcription factor and an amino acid sequence that dimerizes with the other fusion protein of the target interaction; and (b) one fusion protein comprises the transcriptional activator domain of a transcription factor and an amino acid sequence that dimerizes with the other fusion protein of the target interaction, and wherein dimerization between said fusion proteins produces a protein complex that binds to the cis-acting sequence and activates expression of the reporter gene(s) when the target interaction occurs in the host cell; and

(ii) the binding partners of the non-target interaction consist of two fusion proteins, wherein (a) one fusion protein comprises the DNA binding domain of a transcription factor and an amino acid sequence that dimerizes with the other fusion protein of the non-target interaction; and (b) one fusion protein comprises the transcriptional activator domain of a transcription factor and an amino acid sequence that dimerizes with the other fusion protein of the non-target interaction, and wherein dimerization between said fusion proteins produces a protein complex that binds to the cis-acting sequence and activates expression of the reporter gene(s) when the non-target interaction occurs in the host cell.

This embodiment of the invention has versatility in so far as the nature of the binding partners of the target interaction and the non-target interaction are interchangeable, provided that the capability for distinguishing between the target interaction and the non-target interaction is retained.

For example, the fusion proteins of the target interaction and the non-target interaction comprising a transcription activator domain can be the same, or have the same activation properties. In such a case, the fusion proteins of the target and non-target interactions comprising DNA binding domains will be different, as will the cis-acting sequences to which they bind. Alternatively, the proteins of the target or non-target interaction that comprise DNA binding domains may be the same, but fused to different DNA binding domains that bind to different cis-acting elements upstream of the reporter gene(s).

Alternatively, the fusion proteins of the target and non-target interactions comprising DNA binding domains can be the same, or have the same binding affinities. In such a case, the cis-actng sequences of the target and non-target interactions will also be the same or functionally equivalent, however the fusion proteins of the target interaction and the non-target interaction comprising a transcription activator domain will be different.

As will be known to the skilled person, reverse n-hybrid screens require the operable connection of the interaction being assayed to the expression of one a reporter gene in the cell, wherein expression of the reporter is selected against to facilitate the detection of an antagonist of the interaction. The present invention develops this technology further by providing for the selection against the target interaction, concomitant with selection for or against the non-target interaction.

Particularly preferred target interactions susceptible to the inventive method described herein include those interactions between SCL or the dimerization region of SCL and protein selected from the group consisting of: LMO1, LMO2, DRG, mSin3A, E47, a dimerization region of LMO1, a dimerization region of LMO2, a dimerization region of DRG, a dimerization region of mSin3A, and a dimerization region of E47.

Alternatively, the inventive method is capable of detecting the interaction between LMO2 or the dimerization region of LMO2 and a protein selected from the group consisting of: SCL, LMO1, DRG, mSin3A, and E47, a dimerization region of SCL, a dimerization region of LMO1, a dimerization region of DRG, a dimerization region of mSin3A, and a dimerization region of E47.

In a particularly preferred embodiment, the interaction is between the dimerization region of SCL and the dimerizaion region of LMO2 or the dimerization region of E47 or the dimerization region of LMO1.

In an alternative preferred embodiment, the interaction is between the dimerization region of LMO2 and the dimerization region of E47 or the dimerization region of mSin3A.

In a further preferred embodiment, the interaction is between the dimerization region of E47 and the dimerization region of mSin3A.

Preferably, the transcriptional activator domain is the GAL4 activator domain.

Preferably, the DNA binding domain is selected from the group consisting of: LexA operator binding domain, GAL4 DNA binding domain; and cl operator binding domain.

As exemplified herein, the present inventors show that a reverse two hybrid screen of the invention is capable of distinguishing between interactions in the same cell involving (i) SCL and LMO2; and (ii) SCL and the LMO1, mSin3A, or E47 proteins. In additional to distinguishing between these interactions, the method applies reverse n-hybrid screening procedures to identify peptide aptamers that block such interactions, and, as a consequence, have potential utility as therapeutic or diagnostic reagents.

In a further example, the inventive method described herein distinguishes effectively between interactions in the same cell between an RNA molecule and a protein. Accordingly, the present invention is equally applicable to the detection of inhibitors of both protein and nucleic acid interactions in cells.

The improved reverse two hybrid or reverse three hybrid screens described herein require the expression of the following integers in a single cell:

(i) the binding partners of a target interaction such that their interaction enhances the expression of one or more reporter genes in said cell and said expression is partially or completely inhibited by disruption of said target interaction;

(ii) the binding partners of a non-target interaction such that their interaction enhances the expression of one or more reporter genes in said cell and said expression is partially or completely inhibited by disruption of said non-target interaction, wherein said reporter gene(s) is(are) distinct from the reporter gene(s) expressed under control of the target interaction; and

(iii) a candidate peptide being tested for inhibitory activity.

As will be understood by the skilled person, the term “expressing” includes transcription of a gene to produce mRNA with or without any post-transcriptional processing of the primary transcript, such as, for example, mRNA splicing or the addition of a polyadenylate sequence, to produce translatable mRNA. Such “further includes transcription followed by translation of the mRNA, to produce a peptide or protein, with or without any post-translational modification of the protein required to modulate its activity, such as, for example glycosylation, processing, splicing, or transport.

It will be apparent to those skilled in the art from the description herein that the inventive method is equally applicable to the high throughput screening of a panel of candidate peptides for inhibitory activity. For example, a library of clones expressing candidate peptides may be produced and each member of said library introduced into cells in which the target and non-target interactions are capable of being assayed. Those cells in which the target interaction is partially or completely inhibited selected, wherein each selected cell has a clone that inhibits the target interaction in the cell.

Accordingly, as used herein, the term “expressing a candidate peptide”, clearly encompasses the expressing of several members of a library of clones wherein each member expresses a single candidate peptide.

As used herein, the term “library” is a set of diverse nucleotide sequences encoding a set of amino acid sequences, wherein said nucleotide sequences are preferably contained within a suitable vector, cosmid, bacteriophage or virus vector molecule which is suitable for maintenance and/or replication in a cellular host. The term “library” includes a random synthetic peptide library, in which the extent of diversity between the amino acid sequences or nucleotide sequences is numerous, and a limited peptide library in which there is a lesser degree of diversity between said sequences. The term “library” further encompasses diverse amino acid sequences derived from a cellular source, such as, for example, genome fragments obtained for example by shearing or partial digestion of genomic DNA using restriction endonucleases, amongst other approaches. A “peptide library” further includes cells, virus particles and bacteriophage particles comprising the individual amino acid sequences or nucleotide sequences of the diverse set.

Preferred libraries according to this embodiment of the invention are “representative libraries”, comprising a set of amino acid sequences or nucleotide sequences encoding same, which includes all possible combinations of amino acid or nucleotide sequences for a specified length of peptide or nucleic acid molecule, respectively.

The diversity of peptide libraries, in particular those derived from genomic sources, can be increased by means known to those skilled in the art, such as, for example, random or other mutagenesis. In one exemplification of this embodiment, peptide libraries derived from the expression of genomic DNA are amplified or propagated in bacterial strains which are defective in the epsilon (E) subunit of DNA polymerase III (i.e. dnaQ and mutD alleles) and/or are defective in mismatch repair. Escherichia coli mutator strains possessing the mutY and/or mutM and/or mutD and/or mutT and/or mutA and/or mutC and/or mutS alleles are particularly useful for such applications. Bacterial strains carrying such mutations are readily available to those skilled in the art and are fully described for example, by Akiyama et al 1989; Fijalkowska and Schaaper, 1995; Frick et al, 1995; Lu et al, 1995; Maki and Sekiguchi, 1992; Miller, 1992; Miller and Michaels, 1996; Moriya and Gollman, 1992; Schaaper and Comacchio, 1992; Slupska et al, 1996; and Tajiri et al, 1995.

The expressed candidate peptide may comprise any amino acid sequence of at least about 1 to 60 amino acids in length and may be derived from the expression of nucleotide sequences which are prepared by any one of a variety of methods such as, for example, random synthetic generation, or using naturally-occurring genomes as exemplified herein.

Preferably, the peptide is a 20-mer peptide. The use of larger fragments, particularly employing randomly sheared nucleic acid derived from bacterial, yeast or animal genomes, is not excluded.

Alternatively or in addition, the candidate peptide is expressed as a fusion protein with a nuclear targeting motif capable of facilitating targeting of said peptide to the nucleus of said host cell where transcription occurs, in particular the yeast-operable SV40 nuclear localization signal.

Alternatively, or in addition, the candidate peptide is expressed as a fusion protein with a peptide sequence capable of enhancing uptake of the peptide by an isolated cell such as, for example, when the subject peptide is synthesized ex vivo and added to isolated cells in culture. In a preferred embodiment, the peptide sequence capable of enhancing, increasing or assisting penetration or uptake is functional in insect cells or mammalian cells, for example the Drosophila penetratin targeting sequence.

The candidate peptide may also be expressed in a conformationally constrained form. Amino acid sequences which are expressed in a conformationally constrained form may be expressed within a second polypeptide as a fusion protein such that they are effectively “nested” in the secondary structure of the second polypeptide. Alternatively, the peptide, oligopeptide or polypeptide may be circularized within a loop of disulphide bonds to limit conformational diversity, such as, for example, by expressing the peptide within oxidized flanking cysteine residues. This may be particularly beneficial where the amino acid sequences are nested within a surface-exposed or functional site of a protein, such that they are accessible to the interaction of interest. For example, the peptide may be expressed within a thioredoxin (Trx) polypeptide loop.

The binding partners may be expressed from separate DNA molecules or vectors. Alternatively, they can be expressed from a single DNA molecule or vector. The only requirement for expression of the binding partners is that both partners are expressed in a cell at the same time as the candidate peptide being assayed. Additionally, said binding partners are expressed under conditions sufficient to modulate expression of the reporter genes to which they are operably connected in the cell.

The present invention clearly contemplates higher order interactions involving three or four or more binding partners of the target or non-target interaction.

The binding partners are preferably expressed as fusion proteins with a nuclear localization sequence to facilitate their transport to the site of transcription in a eukaryotic cell (i.e. the nucleus), such as, for example, the SV40 large T antigen nuclear localization signal.

The only requirement for a suitable reporter gene is the capability of being expressed in a manner that is readily detected, such as by the phenotype said expression confers on the cell (for example, auxotrophy or prototrophy for a particular metabolite, or conditional lethality in the presence of a particular substrate), or alternatively, by expressing an enzyme activity, or a protein detectable by immunoassay or colorimetric detection, or fluorescence.

Suitable reporter genes include those encoding Escherichia coli β-galactosidase enzyme, the firefly luciferase protein (Ow et al, 1986; Thompson et al, 1991) the green fluorescent protein (Prasher et al, 1992; Chalfie et al, 1994; Inouye and Tsuji, 1994; Cormack et al, 1996; Haas et al, 1996; see also GenBank Accession No. U55762); and the red fluorescent proteins of Discosoma (Matz et al., 1999) or Propionibacterium freudenreichii, (Wildt and Deuschle, 1999). Additionally, the HIS3 gene [Larson, R. C. et al. (1996), Condorelli, G. L. et al. (1996), Hsu, H. L., et al. (1991), Osada, et al. (1995)] and LEU2 gene (Mahajan, M. A. et al., 1996) are also useful.

Reporter genes that are counter selectable reporter genes (i.e. they confer conditional lethality on the cell), or genes that encode fluorescent proteins, or a combination thereof, are particularly preferred. Both types of preferred reporter genes have suitability for high throughput applications, where large numbers of samples are screened in batches. Fluorescence can be readily assayed by fluorometry or fluorescence activated cell sorting (FACS), techniques known to those skilled in the art.

Wherein a reporter gene operably under the control of the target interaction or the non-target interaction encodes a fluorescent protein, detection of reporter gene expression is determined by identifying those cells that fluoresce when said reporter gene is expressed. Alternatively, inhibition of reporter gene expression is determined by identifying those cells that do not fluoresce or have reduced fluorescence compared to when said reporter gene is expressed.

Preferred reporter genes encoding fluorescent proteins include the gfp gene, the cobA gene, and fragments or variants of said gfp gene or cobA gene that encode a fluorescent proteins.

Wherein a reporter gene operably under the control of the target interaction or the non-target interaction is a counter selectable reporter gene that confers conditional lethality on the cell, detection of reporter gene expression is determined by identifying those cells that cease to grow or die when said reporter gene is expressed under conditions that confer lethality. Alternatively, inhibition of reporter gene expression is determined by identifying those cells that do not cease to grow compared to when said reporter gene is expressed, or do not die, under conditions that confer lethality.

Preferred counter selectable reporter genes are those genes that encode a polypeptide capable of converting a non-toxic substrate to a toxic product, in particular a counter selectable reporter gene is selected from the group consisting of: CAN, URA3, CYH2, and LYS2.

In a particularly preferred embodiment, the combination of URA3 and CYH2 genes operably under the control of the target interaction is preferred. Alternatively or in addition, use of the LYS2 gene operably under control of the non-target interaction is particularly preferred.

In an alternative embodiment, multiple reporter genes are operably under the control of the target interaction, wherein said reporter genes comprise at least one counter selectable reporter gene and at least one gene encoding a fluorescent protein such that said detecting comprises identifying those cells grow or survive and do not fluoresce or have reduced fluorescence when said reporter gene is not expressed compared to when said reporter gene is expressed.

Similarly, multiple reporter genes can be placed operably under the control of the non-target interaction which genes include at least one counter selectable reporter gene and at least one gene encoding a fluorescent protein.

Persons skilled in the art will be aware of how to utilize genetic sequences encoding such reporter molecules in performing the invention described herein, without undue experimentation. For example, the coding sequence of the gene encoding such a reporter molecule may be modified for use in the cell line of interest (e.g. human cells, yeast cells) in accordance with known codon usage preferences. Additionally the translational efficiency of mRNA derived from non-eukaryotic sources may be improved by mutating the corresponding gene sequence or otherwise introducing to said gene sequence a Kozak consensus translation initiation site (Kozak, 1987).

To link reporter gene expression to the target interaction, or conversely, to the non-target interaction, the reporter gene should include one or more cis-acting sequences to which one of the binding partners to said interaction can bind or dock. Such features are known to those skilled in the art.

Preferably, the cis-acting sequence of the target interaction or the non-target interaction is selected from the group consisting of: LexA operator, GAL4 binding site, and cl operator. In accordance with this embodiment of the invention, it is preferred for a binding partner that includes a DNA binding domain to be capable of binding to said cis-acting sequence, in which case said DNA binding domain will be selected from the group consisting of: LexA operator binding domain, GAL4 DNA binding domain; and cl operator binding domain.

It will be known to those skilled in the art that the expression of an integer in a cell requires nucleic acid to be placed operably in connection with a promoter sequence that is operable in the cell.

Reference herein to a “promoter” is to be taken in its broadest context and includes the transcriptional regulatory sequences of a classical genomic gene, including the TATA box which is required for accurate transcription initiation in eukaryotic cells, with or without a CCAAT box sequence and additional regulatory elements (i.e. upstream activating sequences, enhancers and silencers). Promoters may also be lacking a TATA box motif, however comprise one or more “initiator elements” or, as in the case of yeast-derived promoter sequences, comprise one or more “upstream activator sequences” or “UAS” elements. For expression in prokaryotic cells such as, for example, bacteria, the promoter should at least contain the −35 box and −10 box sequences.

A promoter is usually, positioned upstream or 5′ of a structural gene, the expression of which it regulates. Furthermore, the regulatory elements comprising a promoter are usually positioned within about 2 kb of the start site of transcription of the gene.

In the present context, the term “promoter” is also used to describe a synthetic or fusion molecule, or derivative which confers, activates or enhances expression of the subject reporter molecule in a cell.

Preferred promoters may contain additional copies of one or more specific regulatory elements, to further enhance expression of the gene and/or to alter the spatial expression and/or temporal expression. For example, regulatory elements which facilitate the enhanced expression of a gene by galactose or glucose or copper may be placed adjacent to a heterologous promoter sequence driving expression of the gene. Promoters comprising regulatory elements of the GAL1 or CUP1 promoters are particularly preferred for titration of the expression of one or more binding partners in response to galactose or copper, respectively, in the culture medium in which the host cell is grown.

Placing a gene operably under the control of a promoter sequence means positioning the said gene such that its expression is controlled by the promoter sequence. Promoters are generally positioned 5′ (upstream) to the genes that they control. In the construction of heterologous promoter/structural gene combinations it is generally preferred to position the promoter at a distance from the gene transcription start site that is approximately the same as the distance between that promoter and the gene it controls in its natural setting, i.e., the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the genes from which it is derived. Again, as is known in the art, some variation in this distance can also occur.

Examples of promoters suitable for use in regulating the expression of the reporter molecule and/or candidate peptide and/or a proteinaceous binding partner include viral, fungal, yeast, insect, animal and plant promoters, especially those that can confer expression in a eukaryotic cell, such as, for example, a yeast cell or a mammalian cell.

Particularly preferred promoters according to the present invention include those naturally-occurring and synthetic promoters which contain binding sites for transcription factors, more preferably for helix-loop-helix (HLH) transcription factors, zinc finger proteins, leucine zipper proteins and the like. Preferred promoters may also be synthetic sequences comprising one or more upstream operator sequences such as, for example, LexA operator sequences or activating sequences derived from any of the promoters referred to herein such as, for example, GAL4 DNA binding sites.

Those skilled in the art will recognise that the choice of promoter will depend upon the nature of the cell being transformed and the molecule to be expressed. Such persons will be readily capable of determining functional combinations of minimum promoter sequences and operators for cell types in which the inventive method is performed.

Whilst the invention is preferably performed in yeast cells, the inventors clearly contemplate modifications wherein the invention is performed entirely in bacterial or mammalian cells, utilizing appropriate promoters which are operable therein to drive expression of the various assay components in such cells. Such embodiments are within the ken of those skilled in the art.

In a particularly preferred embodiment, the promoter is a yeast promoter, mammalian promoter, a bacterial or bacteriophage promoter, selected from the group consisting of: MYC, GAL1, CUP1, PGK1, ADH1, ADH2, PHO4, PHO5, HIS4, HIS5, TEF1, PRB1, GUT1, SPO13, CMV, SV40, LAC, TEF, EM7, SV40, and T7.promoter sequences.

For expression in mammalian cells, it is preferred that the promoter is the CMV promoter sequence, more preferably the CMV-IE promoter or alternatively, the SV40 promoter and, in particular the SV40 late promoter sequence. These promoters, and other promoter sequences suitable for expression of genes in mammalian cells are known in the art.

Examples of mammalian cells contemplated herein to be suitable for expression include COS, VERO, HeLa, mouse C127, Chinese hamster ovary (CHO), WI-38, baby hamster kidney (BHK) or MDCK cell lines, amongst others. Such cell lines are readily available to those skilled in the art.

The prerequisite for producing intact polypeptides in bacterial cells and, in particular, in Escherichia coli cells, is the use of a strong promoter with an effective ribosome binding site, such as, for example, a Shine-Dalgamo sequence, which may be incorporated into expression vectors carrying the first and second nucleotide sequences, or other genetic constructs used in performing the various alternative embodiments of the invention. Typical promoters suitable for expression in bacterial cells such as, for example, E. coli include, but are not limited to, the lacz promoter, temperature-sensitive λ_{L} or λ_{R} promoters, T7 promoter or the IPTG-inducible tac promoter. A number of other vector systems for expressing nucleic acid in E. coli are well-known in the art and are described for example in Ausubel et al (1987) or Sambrook et al (1989). Numerous sources of genetic sequences suitable for expression in bacteria are also publicly available in various vector constructs, such as for example, pKC30 (λ_{L}: Shimatake and Rosenberg, 1981), pKK173-3 (tac: Amann and Brosius, 1985), pET-3 (T7: Studier and Moffat, 1986) or the pQE series of expression vectors (Qiagen, CA), amongst others. Suitable prokaryotic cells for expression include corynebacterium, salmonella, Escherichia coli, Bacillus sp. and Pseudomonas sp, amongst others. Bacterial strains which are suitable for the present purpose are known in the art (Ausubel et al, 1987; Sambrook et al, 1989).

Wherein the promoter is intended to regulate expression of the reporter molecule, it is particularly preferred that said promoter include one or more recognition sequences for the binding of a DNA binding domain derived from a transcription factor, for example a GAL4 binding site or LexA operator sequence.

In accordance with the inventive method, host cells expressing the above components are grown under conditions sufficient to enable the binding partners of the target interaction, at least, to associate. The binding partners of the non-target interaction may also associate under such conditions. However, the formation of the target interaction and the non-target interaction are distinguished by virtue of the operable connection of the target interaction and the non-target interaction to distinct reporter genes, which can be assayed separately or simultaneously, depending upon the reporter genes used. For example, distinct counter selectable reporter genes can be used for assaying the target and non-target interactions, in which case those interactions can be distinguished by survival or growth of cells on particular substrates. As exemplified herein, the present inventors show that it is possible to distinguish between a target interaction operably linked to both URA3 and CYH2 genes, and a non-target interaction linked to the LYS2 gene. In this embodiment, cells in which the target interaction but not the non-target interaction is inhibited are detectable, because they are resistant to fluororotic acid (5-FOA) and cycloheximide, and do not require lysine for growth and/or are sensitive to growth on media containing a-aminoadipate (α-AA). Conversely, cells in which the target interaction but not the non-target interaction activates reporter gene expression are detectable, because they are sensitive to fluororotic acid (5-FOA) and cycloheximide, and require exogenous lysine for growth and/or are resistant to growth on media containing a-aminoadipate (α-AA). Similarly, the present inventors show that it is possible to distinguish between target interaction and non-target interactions operably linked to distinct fluorescent protein-encoding reporter genes, by virtue of detecting the different emission wavelengths of the expressed proteins.

Since the exemplified reporter genes for the non-target interaction pairs are LYS2 and CobA (FIG. 14), specificity of the inhibition achieved by blocking is determined by replica plating clones from the primary screen to media lacking lysine and checking for the presence of red fluorescence under excitation with UV light, indicating no inhibition of the target interaction. The ‘interaction mating’ technique of Finley and Brent (1994) can be used to rapidly determine whether peptide inhibitors isolated from the above screen are specific for the target interaction. However this approach is more time consuming since it requires rescue of the candidate peptide inhibitor plasmids from the diploid strain and re-transformation into a haploid strain of appropriate mating type that lacks the original bait plasmid, prior to mating to strains containing the specificity controls. An advantage of having an endogenous non-target bait linked to a different DNA binding domain is the ability to confirm specificity of the peptide aptamer for a particular interaction directly, in the same strain in which the peptide inhibitor is expressed. This increase in efficiency is important for high through-put applications of the technology.

Preferably, the subject method further comprises selecting and growing the detected cells. Such selection will be based upon the detection of the reporter gene(s) used, and can be readily performed using art-recognized procedures. Similarly, culture methods for growing bacterial, yeast, or mammalian cells are well-known in the art.

Preferably, nucleic acid encoding the candidate peptide, one or more reporter genes, or one or more binding partners, is contained within a vector selected from the group consisting of: pBLOCK-3.0 (SEQ ID NO: 1); pBLOCK-3.2 (SEQ ID NO: 2); pBLOCK-3.4 (SEQ ID NO: 3); pBLOCK-3.6 (SEQ ID NO: 4); pBLOCK-3.8 (SEQ ID NO: 5); pBLOCK-3.9 (SEQ ID NO: 6); pBLOCK-3.10 (SEQ ID NO: 7); pBLOCK-3.11 (SEQ ID NO: 8); pBLOCK-4.0 (SEQ ID NO: 9); and pRT2 (SEQ ID NO: 10). Even more preferably, nucleic acid encoding the candidate peptide is contained within the vector set forth in any one of SEQ ID NOs: 1 to 9; nucleic acid encoding up to three of the binding partners is contained within pBLOCK-3.11 (SEQ ID NO: 8); and nucleic acid encoding two fluorescent reporter genes is contained within the vector pRT2 (SEQ ID NO: 10).

In an alternative embodiment of the present invention, the subject method comprises the further step of introducing into the cellular host one or more further nucleic acid molecules which encodes one or more polypeptide binding partners which are involved in the interaction, placed operably under the control of one or more suitable promoter sequence. Accordingly, the inventive method may be modified by introducing into the cellular host one or more nucleic acids selected from the group consisting of:

(i) nucleic acid encoding a binding partner of the target interaction in an expressible format;

(ii) nucleic acid encoding a binding partner of the non-target interaction in an expressible format;

(iii) nucleic acid encoding the candidate peptide in an expressible format;

(iv) nucleic acid comprising a cis-acting sequence and a reporter gene in an expressible format; and

(v) nucleic acid comprising a cis-acting sequence and a counter selectable reporter gene in an expressible format.

Methods for introducing the nucleic acid into the cellular host include mating those cells having one or more of said nucleic acids so as to combine sufficient nucleic acids into a single cell to select those host cells that grow or survive when the counter selectable reporter genes operably under control of the target interaction are expressed. Alternatively, standard transformation or transfection procedures may be used to introduce individual genes into cells. In fact, any standard means may be used for their introduction, including cell mating, transformation or transfection procedures.

In an alternative embodiment, the present invention provides a method of identifying a peptide that partially or completely inhibits a target interaction between two or more binding partners in a yeast cell but does not inhibit a non-target interaction between some but not all of said binding partners, said method comprising:

(i) transforming a yeast cell with a vector selected from the group consisting of: pBLOCK-3.0 (SEQ ID NO: 1); pBLOCK-3.2 (SEQ ID NO: 2); pBLOCK-3.4 (SEQ ID NO: 3); pBLOCK-3.6 (SEQ ID NO: 4); pBLOCK-3.8 (SEQ ID NO: 5); pBLOCK-3.9 (SEQ ID NO: 6); pBLOCK-3.10 (SEQ ID NO: 7); pBLOCK-3.11 (SEQ ID NO: 8); and pBLOCK-4.0 (SEQ ID NO: 9), wherein said vector further comprises nucleic acid encoding a candidate peptide being tested for inhibitory activity;

(ii) introducing to said transformed yeast cell nucleic acid encoding: (a) the binding partners of said target interaction such that they operably control the expression of one or more counter selectable reporter genes or fluorescent protein-encoding reporter genes in said cellular host, wherein said expression is partially or completely inhibited by disruption of said target interaction; and (b) the binding partners of said non-target interaction such that they operably control the expression of one or more counter selectable reporter genes or fluorescent protein-encoding reporter genes in said cellular host, wherein said expression is partially or completely inhibited by disruption of said non-target interaction and wherein said reporter gene is distinct from the reporter gene(s) expressed under control of the target interaction;

(iii) selecting the recombinants;

(iv) growing the recombinants under conditions sufficient to distinguish the expression of each reporter gene(s) at (a) from expression of the reporter gene(s) at (b); and

(v) detecting those host cells wherein expression of the reporter gene(s) operably under control of the target interaction is(are) partially or completely inhibited and expression of the reporter gene(s) operably under control of the non-target interaction is(are) not inhibited, said detected cells expressing a peptide that partially or completely inhibits the target interaction.

Preferably, the fluorescent protein-encoding reporter genes are introduced to the cell by transforming the cell with the vector pRT2 (SEQ ID NO: 11).

Alternatively, or in addition, nucleic acid encoding one or more of the binding partners of the target interaction and/or the non-target interaction are introduced to the cell by transforming the cell with a derivative of a vector selected from the group consisting of: pBLOCK-3.0 (SEQ ID NO: 1); pBLOCK-3.2 (SEQ ID NO: 2); pBLOCK-3.4 (SEQ ID NO: 3); pBLOCK-3.6 (SEQ ID NO: 4); pBLOCK-3.8 (SEQ ID NO: 5); pBLOCK-3.9 (SEQ ID NO: 6); pBLOCK-3.10 (SEQ ID NO: 7); pBLOCK-3.11 (SEQ ID NO: 8); and pBLOCK-4.0 (SEQ ID NO: 9), wherein said derivative includes a nucleotide sequence encoding said binding partner.

In a preferred embodiment, nucleic acid encoding up to three binding partners of the target interaction and/or the non-target interaction are introduced to the cell by transforming the cell with a derivative of the vector pBLOCK-3.11 (SEQ ID NO: 9), said derivative including nucleotide sequences encoding said binding partners.

In a further preferred embodiment, the subject method further comprises the step of isolating the third nucleic acid molecule from the host cell and sequencing the nucleic acid molecule and deriving the amino acid sequence encoded therefor. Synthetic peptides may be produced, based upon the derived amino acid sequence thus obtained. Techniques for such methods are described, for example by Ausubel et al (1987), amongst others. Those skilled in the art are well versed in such techniques.

In an alternative preferred embodiment, very specific peptides isolated from such screens are expressed, or alternatively, synthesized, prior to their delivery to a culture of eukaryotic cells. The effect of such a peptide on the expression of a range of genes, or on the development of a phenotype, is determined relative to cells lacking the peptide. For example the expression profile of the cell can be monitored using micro-array technology known to those skilled in the art. Accordingly, an inhibitory peptide identified using the improved reverse n-hybrid assay of the invention can serve as a dominant negative probe of a gene pathway. Such pathways are otherwise recalcitrant to analysis by standard genetic means. Additionally, the probes can facilitate the validation of candidate drug targets or the identification of new targets using surrogate markers.

A second aspect of the present invention contemplates a peptide identified by the method of the present invention, wherein said peptides have inhibitory activity against the target interaction, however possess reduced inhibitory activity or no inhibitory activity against the non-target interaction. This aspect of the invention clearly contemplates a pharmaceutical composition comprising said inhibitory peptide and a pharmaceutically acceptable carrier and/or diluent.

Preferably, the peptide antagonizes or inhibits an interaction that produces one or more deleterious effects in eukaryotic cells, in particular human or animal cells. More preferably, the peptides antagonize or inhibit interactions that involve one or more oncoproteins.

The present invention clearly contemplates the use of said inhibitory peptides in the prophylactic or therapeutic treatment of humans or animals. Methods of treatment include their use in peptide therapy regimens such as, for example, in the treatment protocols for patients with leukemia and/or solid tumors. Their use in treatment protocols for said patients includes their administration as a means of blocking further cell division of the malignant cells, for example, targeting of the SCL oncoprotein to arrest the malignant cell division of the patient. The specific targeting of oncoproteins with pharmaceuticals comprising said peptides will reduce the side effects experienced by patients as compared to those experienced with conventional chemotherapy.

Methods of treatment also include other disorders resulting from deleterious expression of aberrant biological molecules that interfere with normal cellular functions.

Pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. Alternatively, injectable solutions may be delivered encapsulated in liposomes to assist their transport across cell membrane. Alternatively or in addition such preparations may contain constituents of self-assembling pore structures to facilitate transport across the cellular membrane. It must be stable under the conditions of manufacture and storage and must be preserved against the contaminating/destructive action of microorganisms such as, for example, bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as, for example, lecithin, by the maintenance of the required particle size in the case of dispersion and by the use of surfactants. Preventing the action of microorganisms in the compositions of the invention is achieved by adding antibacterial and/or antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and freeze-drying, to yield a powder of the active ingredient plus any additional desired ingredient from previously sterile-filtered solution thereof.

When the active ingredients are suitably protected they may be orally administered, for example, with an inert diluent or with an edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that a dosage unit form contains between about 0.1 ug and 20 g of active compound.

The tablets, troches, pills, capsules and the like may also contain binding agents, such as, for example, gum, acacia, corn starch or gelatin. They may also contain an excipient, such as, for example, dicalcium phosphate. They may also contain a disintegrating agent such as, for example, corn starch, potato starch, alginic acid and the like. They may also contain a lubricant such as, for example, magnesium stearate. They may also contain a sweetening agent such a sucrose, lactose or saccharin. They may also contain a flavoring agent such as, for example, peppermint, oil of wintergreen, or cherry flavoring.

When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparaben as preservatives, a dye and flavoring such as, for example, cherry or orange flavor. Of course, any material used in preparing any dosage unit form should be pharmaceutically pure and substantially non-toxic in the amounts employed. In addition, the active compound(s) may be incorporated into sustained-release preparations and formulations.

The present invention also extends to forms suitable for topical application such as, for example, creams, lotions and gels.

Pharmaceutically acceptable carriers and/or diluents include any and all solvents, dispersion media, coatings, antibacterials and/or antifungals, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated.

Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated, each unit containing a predetermined quantity of active material calculated to produce the desired therapeutic effect in association with the required pharmaceutical carrier. The dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active 35 compound is generally present in from about 0.5 pg to about 2000 mg/ml of carrier. In the case of compositions containing supplementary active ingredients, the dosages are determined by reference to the usual dose and manner of administration of-the said ingredients.

The pharmaceutical composition may also comprise genetic molecules such as, for example, a vector capable of transfecting target cells where the vector carries a nucleic acid molecule capable of inhibiting such deleterious biological interactions. The vector may, for example, be a viral vector.

A further aspect of the present invention provides a vector for expressing one or more peptides and/or binding partners in a prokaryotic or eukaryotic cell, said vector comprising the features of a vector selected from the group consisting of: pBLOCK-3.0 (SEQ ID NO: 1); pBLOCK-3.2 (SEQ ID NO: 2); pBLOCK-3.4 (SEQ ID NO: 3); pBLOCK-3.6 (SEQ ID NO: 4); pBLOCK-3.8 (SEQ ID NO: 5); pBLOCK-3.9 (SEQ ID NO: 6); pBLOCK-3.10 (SEQ ID NO: 7); pBLOCK-3.11 (SEQ ID NO: 8); and pBLOCK4.0 (SEQ ID NO: 9). The specific characteristics of such shuttle vectors will be apparent to the skilled person from the detailed description of the invention and the accompanying drawings and Sequence Listing.

A further aspect of the present invention provides a vector for expressing multiple fluorescent reporter genes in a yeast cell, said vector comprising:

(i) a green fluorescent protein expression cassette comprising the gfp gene operably under control of a chimeric yeast operable LexA/GAL1 promoter having multiple LexA operator sites; and

(ii) a red fluorescent protein expression cassette comprising the cobA gene operably under control of a chimeric cl/GAL1 promoter having multiple cl operator sites.

The specific characteristics of such a vector will be apparent to the skilled person from the detailed description of the invention and the accompanying drawings and Sequence Listing.

It will be apparent from the description herein that the efficacy of any peptides identified by the reverse n-hybrid screens of the invention can be validated in several ways, and generally, by comparing the effect of the peptides on target cells of interest. For example, the exclusive rescue of yeast peptide library plasmids from positive blocking clones is rapidly achieved through selection in bacteria for the unique marker on the pBLOCK vector (e.g. blasticidin resistance). The DNA encoding the peptide can then be sequenced. For peptide libraries derived from sequenced natural genomes, it is not necessary to sequence the inhibitory peptide clone. Instead, the positive clones are merely transferred, such as by robot, onto master stocks, and the peptide-encoding DNA inserts amplified using PCR, to yield probes that can be hybridized to oligonucleotide micro arrays. Such arrays represent the whole genomes used to construct the library. The genomic location of a given candidate blocker can then be deduced by reference to the coordinates of the DNA micro array. The rescued DNA is then expressed in a suitable cell line to assess its effect on the phenotype or the expression profile of the cell. Such information is useful to confirm the inhibitory activity, therapeutic activity, or prophylactic activity, of the peptide in vivo, or to identify new binding partners, or drug targets in the same biochemical/signalling pathways as the partner of the target interaction.

Accordingly, a further aspect of the invention is directed to a method for determining the effect of a peptide on a eukaryotic cell comprising:

(i) isolating a nucleotide sequence encoding a peptide inhibitor identified by the method described herein;

(ii) transfecting said eukaryotic cell with the isolated nucleic acid; and

(iii) comparing the phenotype or expression pattern of the transfected eukaryotic cell to the phenotype or expression pattern of an otherwise isogenic non-transfected cell, wherein a different phenotype or expression pattern indicates that the peptide has an effect on the cell.

Preferably, this aspect of the invention is performed on a mammalian cell.

The expression pattern of the transfected eukaryotic cell may be compared to the expression pattern of an otherwise isogenic non-transfected cell, by producing an array of protein or nucleic acid expressed by the transfected and non-transfected cells and comparing said protein or nucleic acid. Array technologies offer a considerable advantage in terms of providing rapid high throughput readouts of data, thereby facilitating the identification of novel binding partners for the inhibitory peptide, which binding partners may be nucleic acid or protein. Moreover, the novel binding partners may also be previously unknown binding partners for one or more of the binding partners of the target interaction assayed in accordance with the inventive method described herein.

Furthermore, when this method is performed on a diseased cell, such as, for example, a leukemia cell or other cancer cell, information derived from the modified expression pattern of the transfected cell, or the modified phenotype of said cell, is useful for designing new prophylactic or therapeutic drugs. In particular, the capability of a particular peptide to partially or completely reverse or correct the diseased phenotype (e.g. cancerous phenotype) can be determined.

Accordingly, a further aspect of the invention provides a process for identifying a peptide for the prophylactic or therapeutic treatment of a mammal comprising:

(i) isolating a nucleotide sequence encoding a peptide inhibitor identified by the method described herein;

(ii) transfecting said eukaryotic cell with the isolated nucleic acid;

(iii) comparing the phenotype or expression pattern of the transfected eukaryotic cell to the phenotype or expression pattern of an otherwise isogenic non-transfected cell, wherein a different phenotype or expression pattern indicates that the peptide has an effect on the cell; and

(iv) selecting a peptide that reverts the phenotype or expression profile of the transfected cell.

### BRIEF DESCRIPTION OF THE DRAWINGS

FIG. 1 is a schematic representation of a generalized reverse two hybrid screen for peptides that specifically antagonize the interaction between two hypothetical proteins (designated X and Y in the Figure), however do not block the interaction between one of said hypothetical proteins (designated X in the figure) and another hypothetical protein (designated Z in the figure).

Panel A shows the expected expression profile and selection characteristics of the cell in the absence of antagonism of the target X/Y interaction and the non-target X/Z interaction. The “prey” (designated “Prey-X” plus “AD” in the figure) for both the target interaction (top two rows) and the non-target interaction (lower row) comprises protein X expressed as a fusion protein with the activation domain (AD) of a transcription factor. The “bait” for the target interaction (top two rows) comprises a fusion protein between protein Y and the DNA binding domain of the LexA protein (designated “LexA-Y” in the figure). The reporter genes for the target interaction (top two rows) consist of chimeric counter selectable reporter genes URA3 and CYH2, and the green fluorescent protein-encoding gfp gene (“GFP”; Prasher et al., 1992; Chalfie et al., 1994; Inouye and Tsuji, 1994) comprising one or more LexA operator sequences (LexAop) to which the bait can bind. The non-target bait comprises a fusion protein between protein Z and the cl repressor protein (stippled box labeled “cl-Z”). The reporter gene for the non-target interaction consists of chimeric LYS2 gene and chimeric cobA gene, each comprising one or more cl operator sequences (stippled boxes labeled “clop”) to which the non-target bait can bind. The cobA gene encodes a red fluorescent protein (Wildt and Deuschle, 1999). Black flags with skull and cross-bones indicate expression of the counter selectable reporter genes. Sun symbols indicate expression of the gfp and cobA reporter genes encoding green and red fluorescent proteins, respectively. Accordingly, the yeast strain is sensitive to 5-FOA and cycloheximide, due to the activating interaction between the target bait and prey fusion proteins bound to the LexA operator site(s) of the counter selectable reporter genes URA3 and CYH2; and prototrophic for lysine biosynthesis, due to the activating interaction between the non-target bait and prey fusion proteins bound to the cl operator sites (clop) of the LYS2 reporter gene which prototrophy can complement an auxotrophic Iys2 mutation in yeast. The strain can also express the gfp and cobA genes under the control of distinct upstream promoter elements, and, as a consequence, fluoresces at wavelengths for green light and/or red light. Activation of these non-lethal reporter genes can be used to quantitate the strength of reporter gene activation using non-counterselectable conditions.

Panel B shows the expected expression profile and selection characteristics of the cell in the presence of an inhibitory peptide (protein marked with arrow indicating “peptide inhibitor”) that specifically blocks the X/Y target interaction (top two rows), but not the non-target X/Z interaction (lower row). Features of the reporter genes and binding partners are as described above. Sun symbols indicate expression of the cobA reporter gene encoding a red fluorescent protein. As shown, the inhibitory peptide (peptide inhibitor) prevents interaction between the bait of the target interaction (LexA-Y) and prey (Prey-X/AD), such that there is no expression in the cell of the URA3 or CYH2 counter selectable reporter genes, or the gfp reporter gene, of the target X/Y interaction. Accordingly, the yeast strain is made resistant to 5-FOA and cycloheximide, however no longer fluoresces green. Additionally, the inhibitory peptide (peptide inhibitor) fails to inhibit the interaction between the prey (Prey-X/AD) and the bait of the non-target interaction (cl-Z), and as a consequence, the cells remain prototrophic for lysine biosynthesis, due to the activating interaction between the non-target bait and prey fusion proteins bound to the cl operator sites (clop) of the LYS2 reporter gene which prototrophy can complement an auxotrophic lys2 mutation in yeast. The strain also continues to express the cobA gene, and, as a consequence, fluoresces red.

FIG. 2 is a schematic representation of a reverse two hybrid screen for peptides that specifically antagonize the SCL/LMO2 interaction in yeast cells, however do not block the SCL/E47 interaction.

Panel A shows the expected expression profile and selection characteristics of the cell in the absence of antagonism of the target SCL/LMO2 interaction and the non-target SCL/E47 interaction. The “prey” for both the target interaction (top row) and the non-target interaction (lower row) comprises SCL expressed as a fusion protein with the activation domain (Act) of a transcription factor (i.e. joined boxes marked SCL and Act). The “bait” for the target interaction (top row) comprises a fusion protein between LMO2 and the DNA binding domain of the LexA protein (i.e. joined boxes marked LMO2 and LexA). The reporter genes for the target interaction (top row) consist of chimeric URA3 and CYH2 genes comprising one or more LexA operator sequences to which the bait can bind. The non-target bait comprises a fusion protein between E47 (box marked E47) and the cl repressor protein (circle labeled cl). The reporter gene for the non-target interaction consists of a chimeric LYS2 gene comprising one or more cl operator sequences to which the non-target bait can bind. Arrows indicate expression of the reporter genes. Accordingly, the yeast strain is sensitive to 5-FOA and cycloheximide, due to the activating interaction between the target bait and prey fusion proteins bound to the LexA operator site(s) of the counter selectable reporter genes URA3 and CYH2; and prototrophic for lysine biosynthesis, due to the activating interaction between the non-target bait and prey fusion proteins bound to the cl operator sites (clop) of the LYS2 reporter gene which prototrophy can complement an auxotrophic lys2 mutation in yeast.

Panel B shows the expected expression profile and selection characteristics of the cell in the presence of an inhibitory peptide (Δ) that specifically blocks the SCL/LMO2 target interaction (top row), but not the non-target SCL/E47 interaction (lower row). Features of the reporter genes and binding partners are as described above. Arrows indicate expression of the reporter genes. Blunt-ended lines (}) indicate an absence of detectable reporter gene expression. Accordingly, following introduction of an inhibitory peptide aptamer into the cell of panel A, the phenotype of the strain is converted to 5-FOA-resistant, and cycloheximide-resistant, due to inhibition of the activating interaction between the target bait bound to the LexA operator site(s) of the counter selectable reporter genes (URA3 and CYH2) and the prey fusion protein; and prototrophic for lysine biosynthesis, due to the activating interaction between the non-target bait and prey fusion proteins bound to the cl operator sites (clop) of the LYS2 reporter gene, which prototrophy can complement an auxotrophic lys2 mutation in yeast.

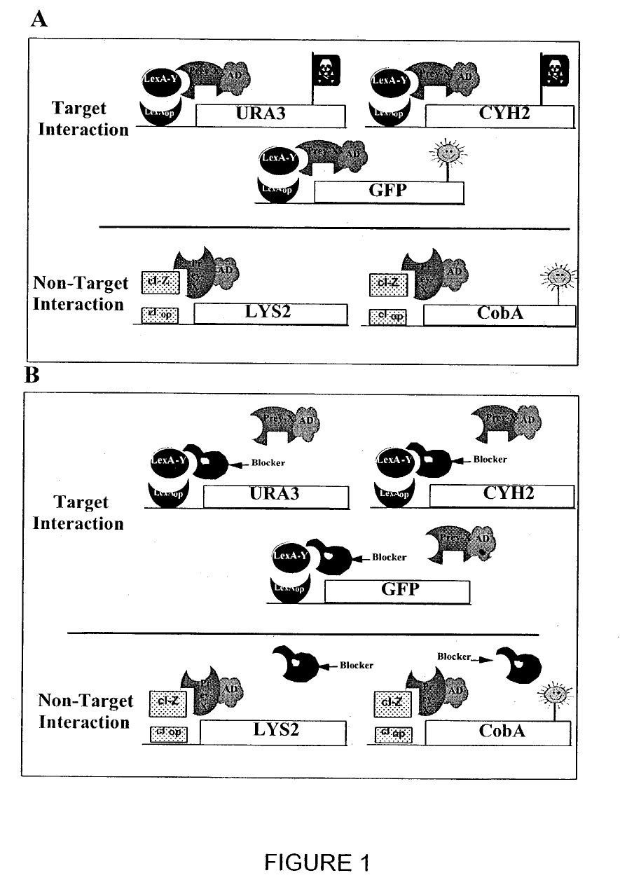

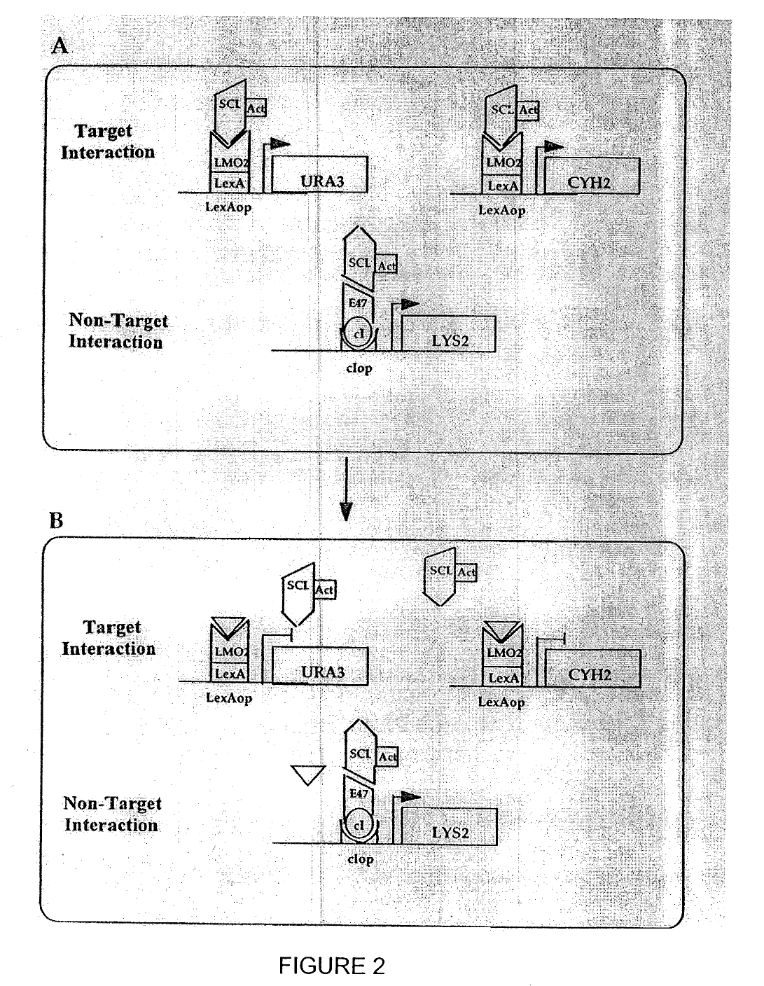

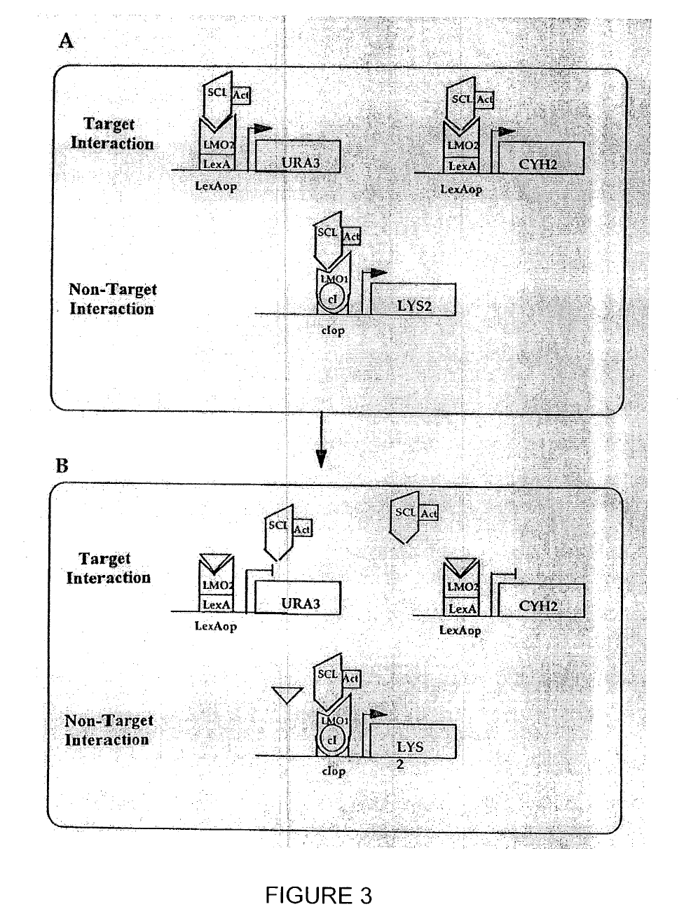

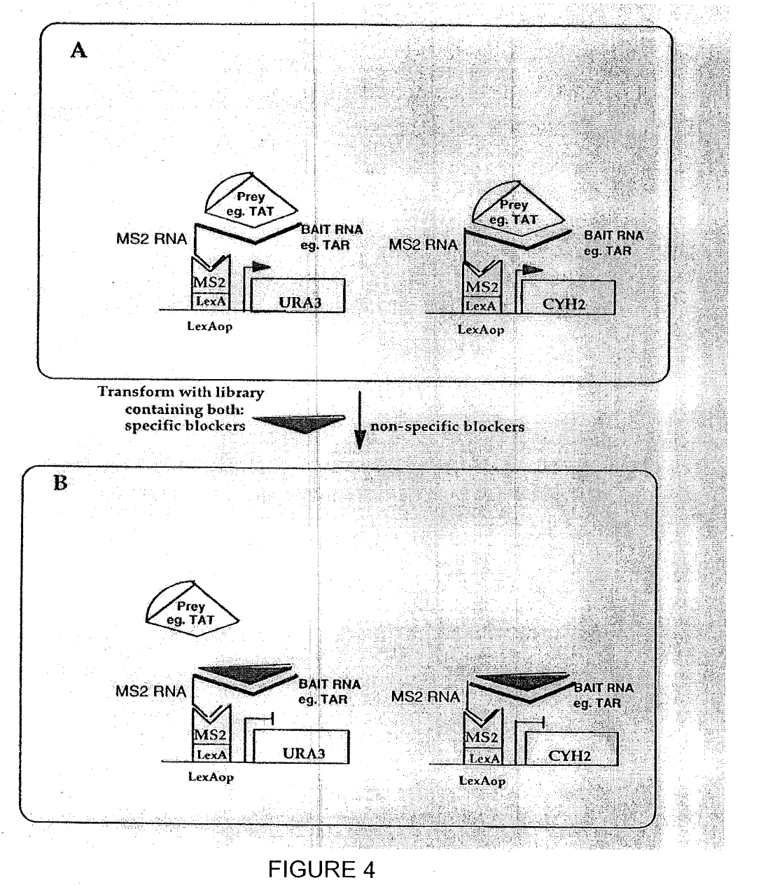

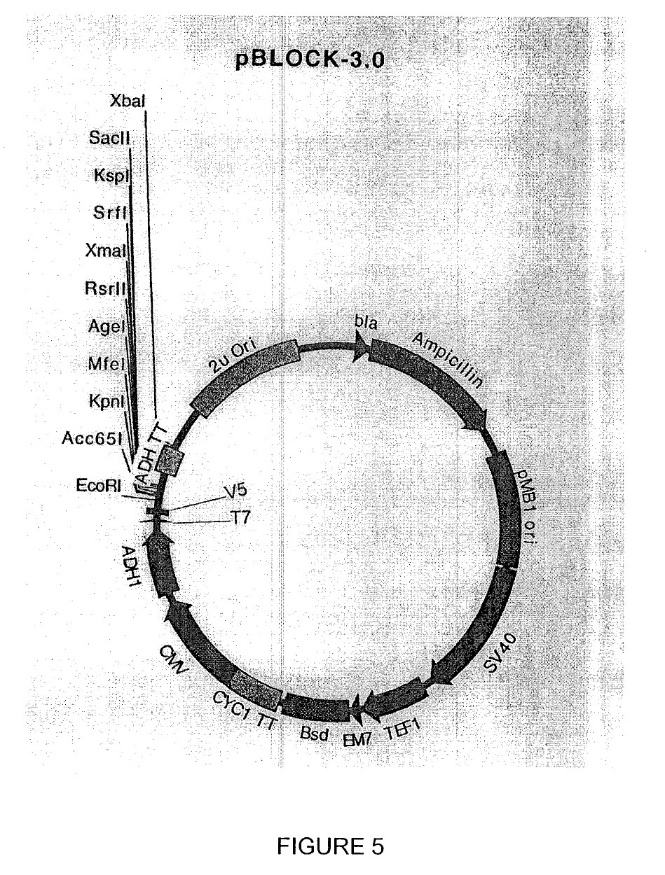

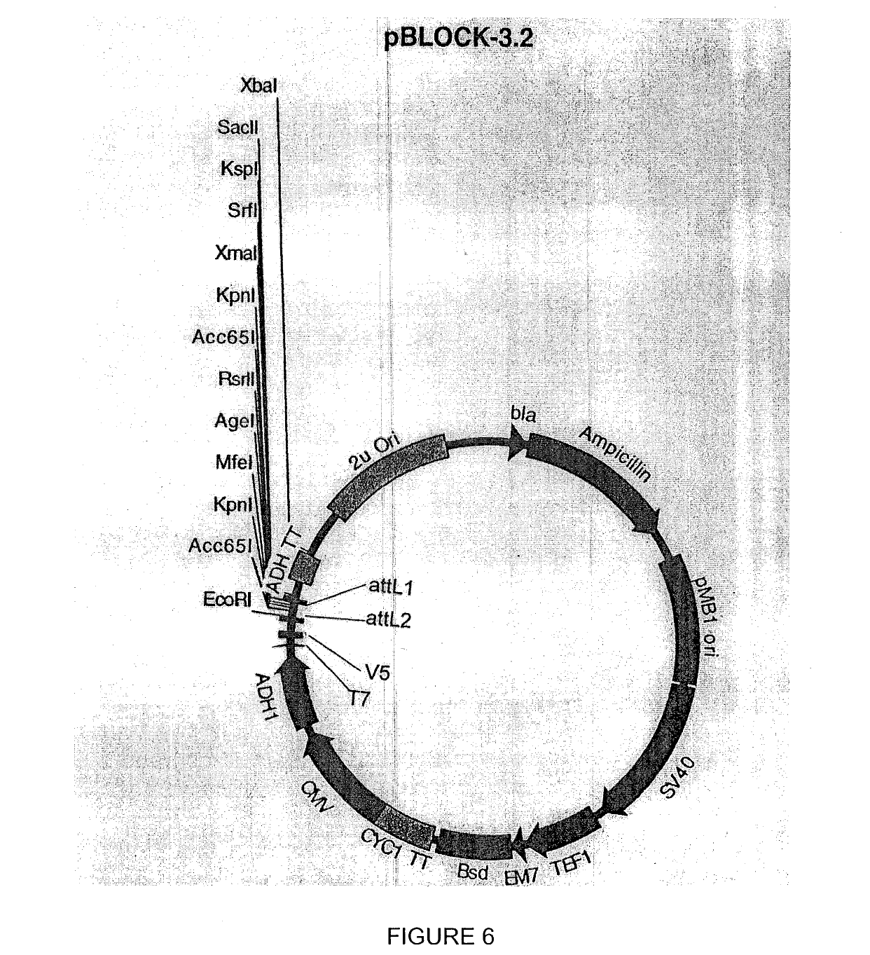

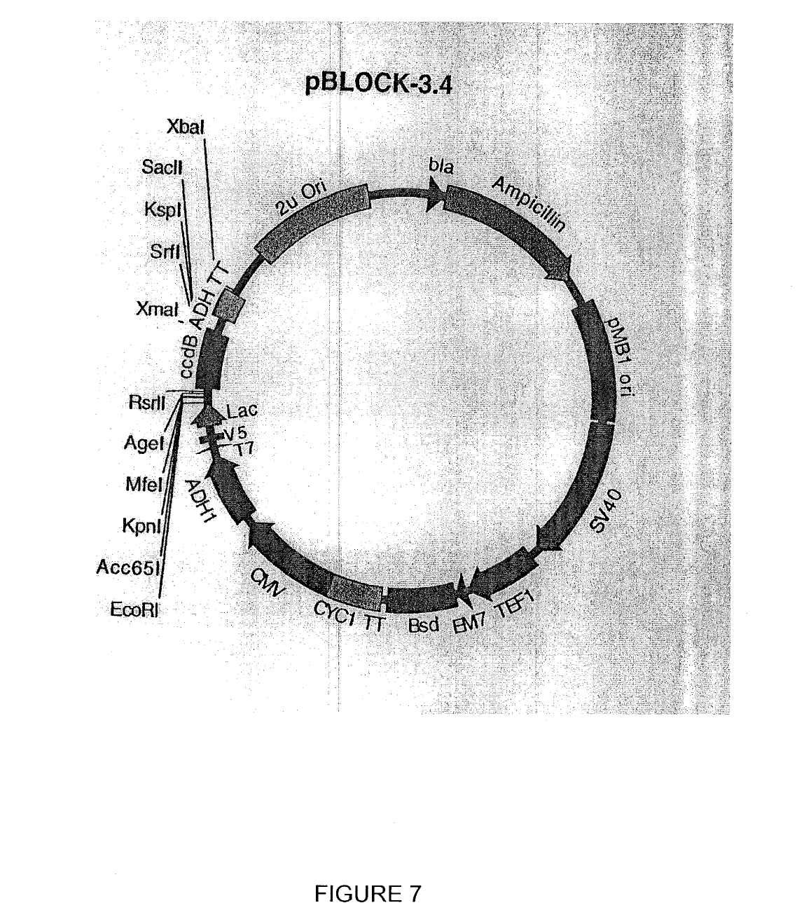

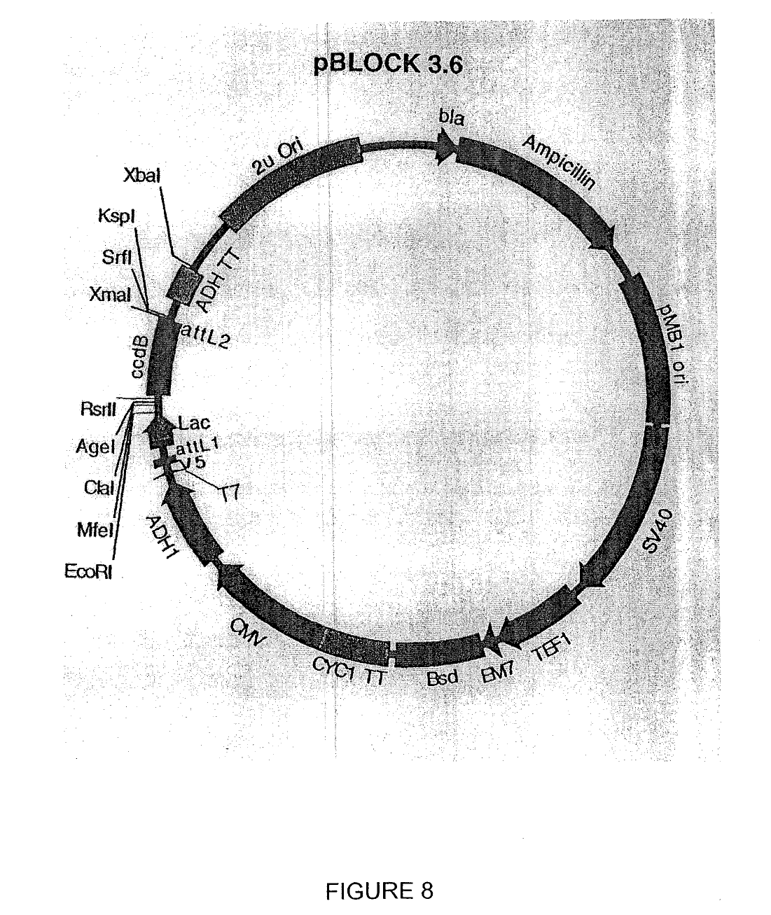

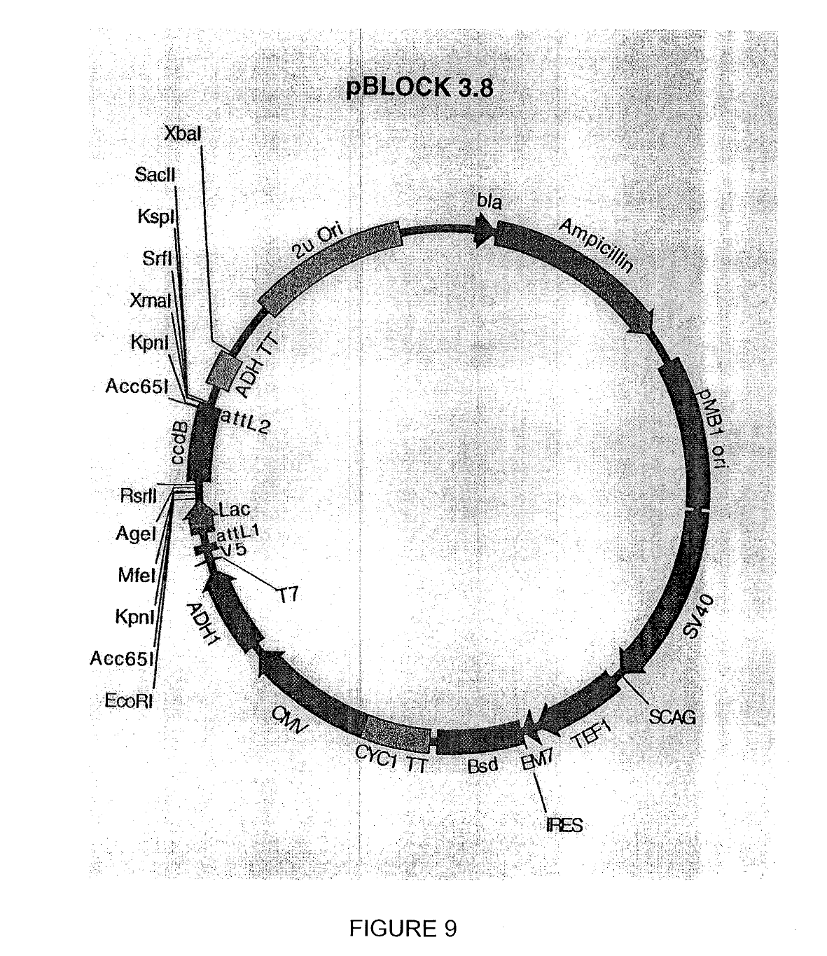

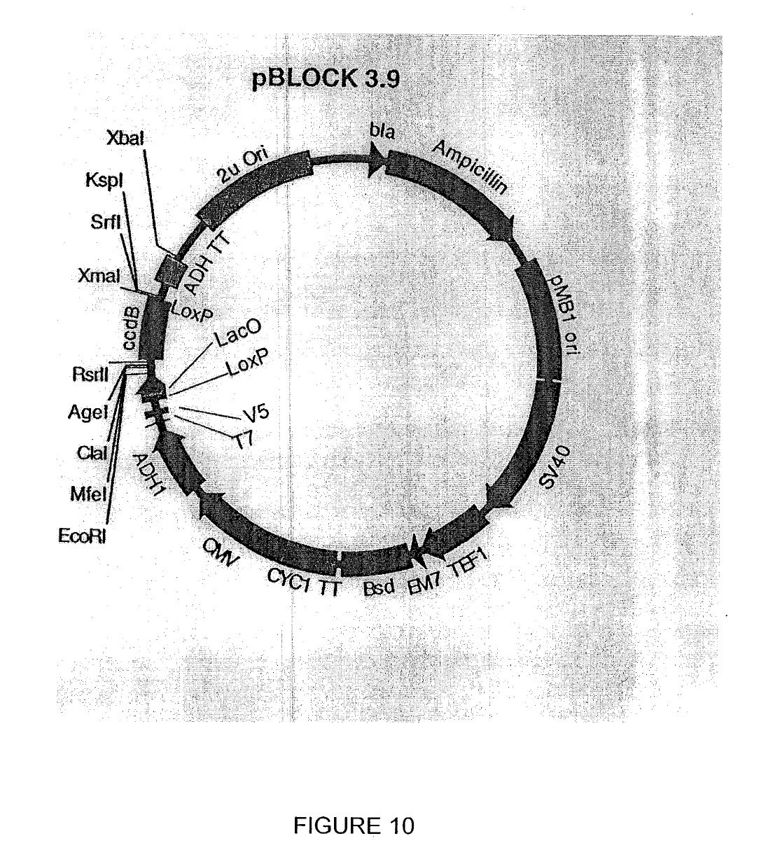

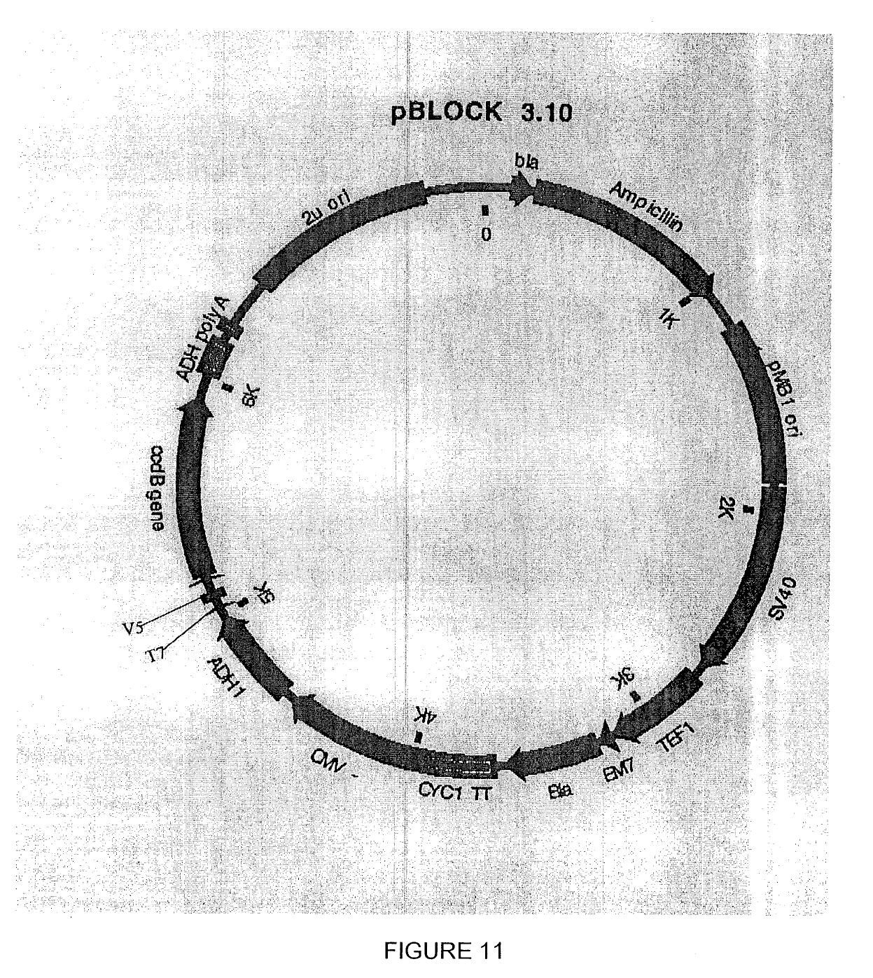

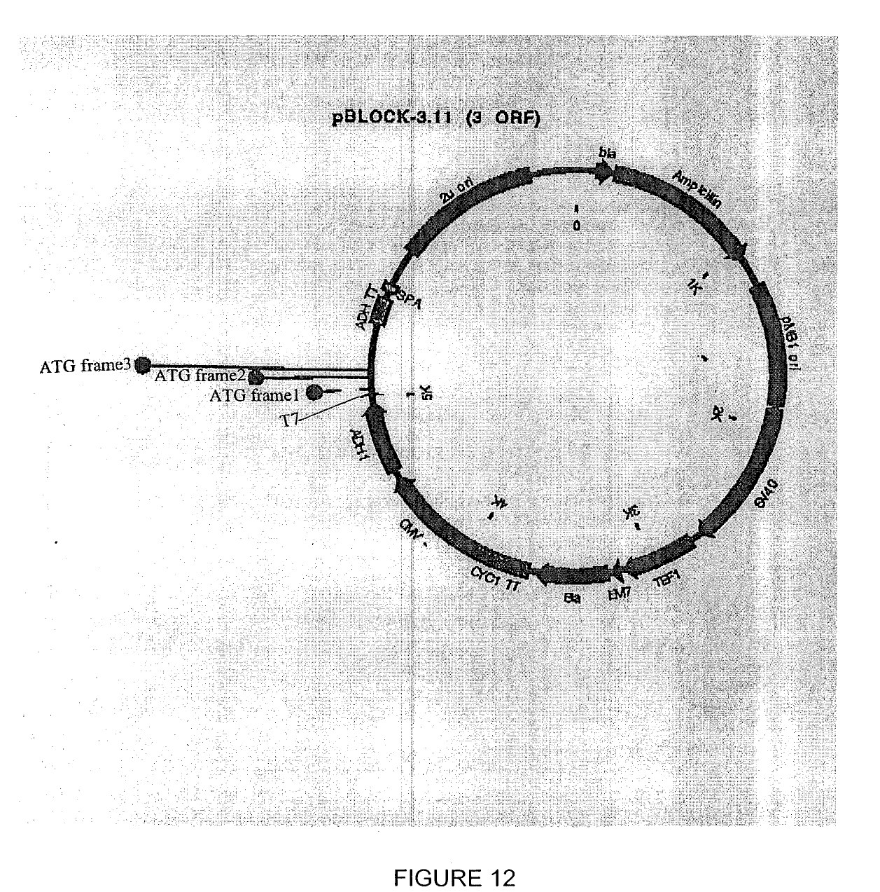

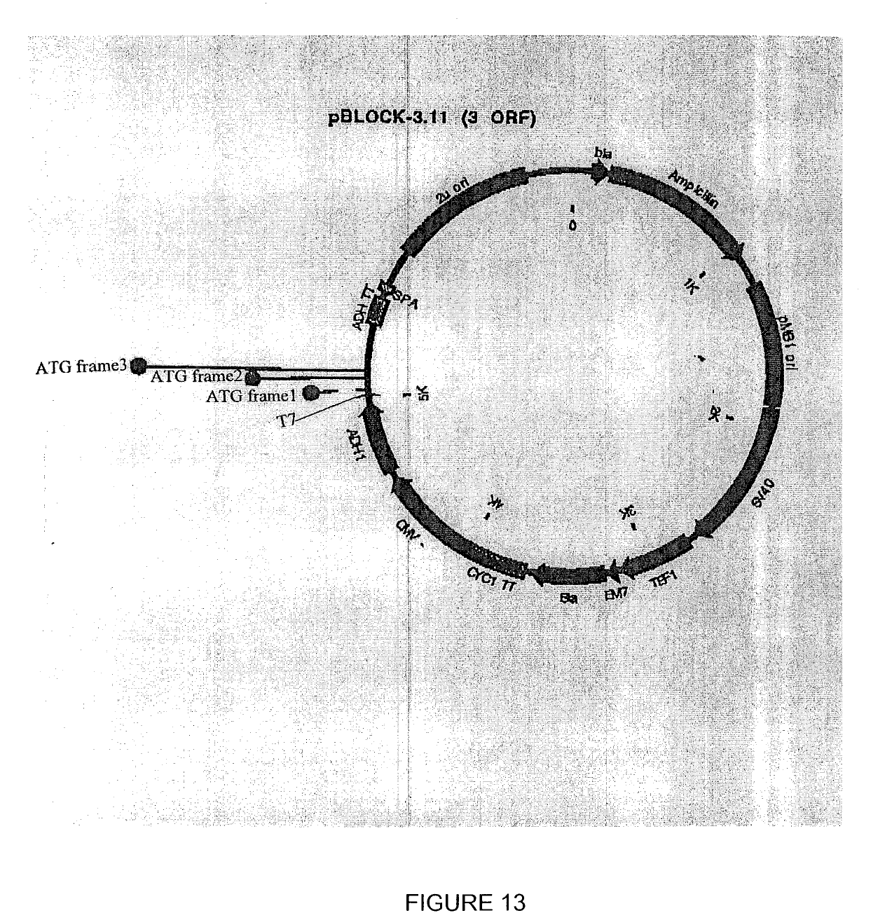

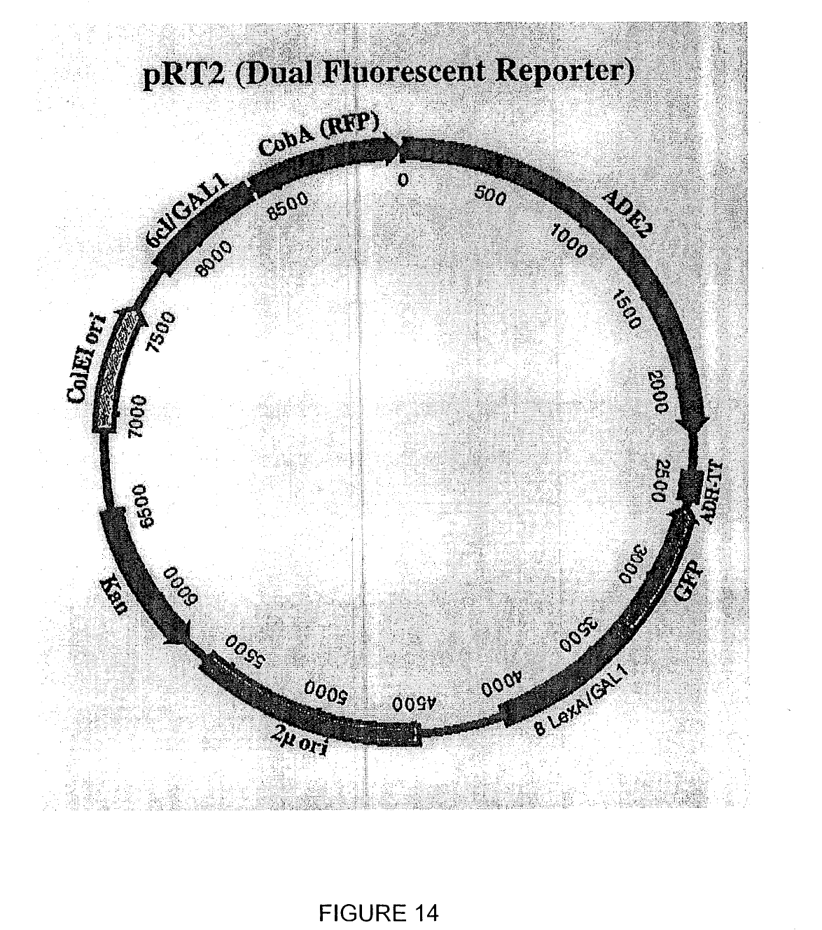

FIG. 3 is a schematic representation of a reverse two hybrid screen for peptides that specifically antagonize the SCL/LMO2 interaction in yeast cells, however do not block the SCL/LMO1 interaction. Panel A shows the expected expression profile and selection characteristics of the cell in the absence of antagonism of the target SCL/LMO2 interaction and the non-target SCL/E47 interaction. The “prey” for both the target interaction (top row) and the non-target interaction (lower row) is as described for FIG. 1 supra. The “bait” and reporter genes for the target interaction (top row), and the reporter gene for the non-target interaction (lower row), are also as described for FIG. 1 supra. The non-target bait comprises a fusion protein between LMO1 (box marked LMO1) and the cl repressor protein (circle labeled cl). Arrows indicate expression of the reporter genes. Accordingly, the yeast strain is sensitive to 5-FOA and cycloheximide, due to the activating interaction between the target bait and prey fusion proteins bound to the LexA operator site(s) of the counter selectable reporter genes URA3 and CYH2; and prototrophic for lysine biosynthesis, due to the activating interaction between the non-target bait and prey fusion proteins bound to the cl operator sites (clop) of the LYS2 reporter gene which prototrophy can complement an auxotrophic lys2 mutation in yeast.

Panel B shows the expected expression profile and selection characteristics of the cell in the presence of an inhibitory peptide (Δ) that specifically blocks the SCL/LMO2 target interaction (top row), but not the non-target SCL/LMO1 interaction (lower row). Features of the reporter genes and binding partners are as described above and in the legend to FIG. 1. Arrows indicate expression of the reporter genes. Blunt-ended lines (}) indicate an absence of detectable reporter gene expression. Accordingly, following introduction of an inhibitory peptide aptamer into the cell of panel A, the phenotype of the strain is converted to 5-FOA-resistant, and cycloheximide-resistant, due to inhibition of the activating interaction between the target bait bound to the LexA operator site(s) of the counter selectable reporter genes (URA3 and CYH2) and the prey fusion protein; and prototrophic for lysine biosynthesis, due to the activating interaction between the non-target bait and prey fusion proteins bound to the cl operator sites (clop) of the LYS2 reporter gene, which prototrophy can complement an auxotrophic lys2 mutation in yeast.

FIG. 4 is a schematic representation of a reverse three hybrid screen for peptides that specifically inhibit the interaction between nucleic acid and protein in yeast cells.

Panel A shows the expected expression profile and selection characteristics of the cell in the absence of antagonism of the target interaction. The non-target interaction is not indicated in the Figure. The “prey” for both the target interaction (and any non-target interaction, not indicated) is an RNA binding protein (TAT) expressed as a fusion protein with the activator domain of a transcription factor. The assay employs two “baits” for the target interaction comprising the following: (i) a hybrid MS2/TAR RNA molecule comprising at least the protein-binding regions of the MS2 coat protein-encoding RNA and TAR-encoding RNA that binds to TAT; and (ii) a fusion protein between MS2 and the DNA binding domain of the LexA protein (i.e. joined boxes marked MS2 and LexA). The reporter genes for the target interaction consist of chimeric URA3 and CYH2 genes comprising one or more LexA operator sequences to which the bait can bind. The non-target bait (not shown) comprises a fusion protein between any other protein to which the MS2/TAR hybrid RNA can bind, (either through the MS2-binding nucleotide sequence moiety or the TAT-binding sequence) and the cl repressor protein. The reporter gene for the non-target interaction (not shown) consists of a chimeric LYS2 gene comprising one or more cl operator sequences to which the non-target bait can bind. Arrows indicate expression of the reporter genes. Accordingly, the yeast strain is sensitive to 5-FOA and cycloheximide, due to the interaction between the target baits (LexA/MS2 protein and MS2/TAR RNA) and the prey fusion protein (i.e. the TAT-activator fusion) at the LexA operator site(s), which interaction activates expression of the counter selectable reporter genes URA3 and CYH2. Additionally, the strain is prototrophic for lysine biosynthesis, due to the interaction between the non-target bait and prey fusion proteins bound to the cl operator sites (not shown), which interaction activates the expression of the LYS2 reporter gene.

Panel B shows the expected expression profile and selection characteristics of the cell in the presence of an inhibitory peptide (▾) that specifically blocks the RNA/protein target interaction, but not the non-target RNA/protein interaction (not shown). Features of the reporter genes and binding partners are as described above. Blunt-ended lines (}) indicate an absence of detectable reporter gene expression. Accordingly, following introduction of an inhibitory peptide aptamer into the cell of panel A, the phenotype of the strain is converted to 5-FOA-resistant, and cycloheximide-resistant, due to inhibition of the activating interaction between the prey TAT-activator fusion protein and the target MS2/TAR bait RNA, said inhibition preventing expression of the counter selectable reporter genes (URA3 and CYH2). The strain is also prototrophic for lysine biosynthesis, due to the continued activating interaction between the non-target baits and the prey fusion protein at the cl operator sites of the LYS2 reporter gene.

FIG. 5 is a schematic representation of the vector pBLOCK-3.0 (SEQ ID NO: 1) for expressing a peptide in a cell comprising:

(i) an expression cassette comprising: (a) a multiple cloning site having the restriction sites EcoRI, Acc651, KpnI, MfeI, AgeI, RsrII, XmaI, SrfI, KspI, and SacII, for insertion of a nucleotide sequence encoding said peptide, wherein said multiple cloning site is adjacent to a sequence encoding the V5 epitope tag and SV40 nuclear localization sequence (not shown) such that a fusion polypeptide comprising said peptide and said epitope tag and nuclear localization sequence can be expressed; (b) the tandem promoters T7, ADH1, and CMV, for regulating expression of said fusion polypeptide in the cells of bacteria, yeast, and mammals, respectively; and (c) a terminator sequence (ADH TT) between said multiple cloning site and an Xbal site, and distal to said tandem promoters;

(ii) a selectable marker gene (Bsd) for conferring resistance to basticidin in yeast and bacteria operably under the control of tandem yeast and bacterially-operable promoters TEF1 and EM7, and placed upstream of the yeast-operable transcription terminator CYC1 (CYC TT);

(iii) a selectable marker gene (ampicillin) for conferring resistance to the antibiotic ampicillin in bacteria operably under the control of the bla promoter;

(iv) a bacterial origin of replication (pMB1 ori); and

(v) a eukaryotic origin of replication (2 p Ori).

FIG. 6 is a schematic representation of the vector pBLOCK-3.2 (SEQ ID NO: 2) for expressing a peptide in a cell comprising the features of pBLOCK-3.0 (SEQ ID NO: 1) as described in the legend to FIG. 5, except that the multiple cloning site has the restriction sites EcoRI, Acc651, KpnI, MfeI, AgeI, RsdI, Acc651, KpnI, XmaI, SrfI, KspI, and SacII, and is flanked by bacteriophage A integration sites (attL1 and attL2) adjacent to a sequence encoding the V5 epitope tag such that a fusion polypeptide comprising said peptide and said epitope tag is capable of being expressed and nucleic acid encoding the peptide (not encoding said fusion polypeptide) is subsequently able to be excised by homologous recombination about said integration sites.

FIG. 7 is a schematic representation of the vector pBLOCK-3.4 (SEQ ID NO: 3) for expressing a peptide in a cell comprising the features of pBLOCK-3.0 (SEQ ID NO: 1) as described in the legend to FIG. 5, except that the expression cassette comprises the counter selectable reporter gene ccdB cloned between the RsdI and XmaI sites of the multiple cloning site, and in operable connection with a lac promoter placed upstream of the EcoRI-RsrII portion of the multiple cloning site. Accordingly, the vector is conditionally lethal in bacterial cells, unless the chimeric Lac-ccdB gene is disrupted by insertion of nucleotide sequences encoding the peptide to be expressed, within the EcoRI-RsrII portion of the multiple cloning site.

FIG. 8 is a schematic representation of the vector pBLOCK-3.6 (SEQ ID NO: 4) for expressing a peptide in a cell, which comprises the features of pBLOCK-3.4 (SEQ ID NO: 3) as described in the legend to FIG. 7, except that (i) the multiple cloning site for inserting the pepbde-encoding sequence has the unique restriction sites EcoRI, MfeI, ClaI, AgeI and RsrII; and (ii) the chimeric lac-ccdB gene is flanked by bacteriophage λ integration sites (attL1 and attL2) between the sequence encoding the V5 epitope tag (V5) and the ADH terminator (ADSH TT) to facilitate excision.

FIG. 9 is a schematic representation of the vector pBLOCK-3.8 (SEQ ID NO: 5) for expressing a peptide in a cell, which vector comprises the features of pBLOCK-3.6 (SEQ ID NO: 4) as described in the legend to FIG. 8, and further includes: (i) a single chain antibody-encoding gene (SCAG) placed operably under the control of the SV40 promoter; and (ii) an internal ribosome entry site sequence (IRES) placed between the EM7 promoter and the Bsd gene.

FIG. 10 is a schematic representation of the vector pBLOCK-3.9 (SEQ ID NO: 6) for expressing a peptide in a cell, which vector comprises the features of pBLOCK-3.6 (SEQ ID NO: 4) as described in the legend to FIG. 8, however the bacteriophage A integration sites (attL1 and attL2) of pBLOCK-3.6 are replaced by LoxP sites to facilitate Cre/loxP-mediated excision of the chimeric lac-ccdB gene.

FIG. 11 is a schematic representation of the vector pBLOCK-3.10 (SEQ ID NO: 7) for expressing a peptide in a cell, which vector comprises the features of pBLOCK-3.4 (SEQ ID NO: 3) as described in the legend to FIG. 7.

FIG. 12 is a schematic representation of the vector pBLOCK-3.11 (SEQ ID NO: 8) for simultaneously expressing up to three distinct peptides in the same cell and from the same vector, which vector comprises the features of pBLOCK-3.0 (SEQ ID NO: 1) as described in the legend to FIG. 5, however contains translation start ATG codons linked in-frame to sequences encoding the epitope and SV40 nuclear localization domains, in each of the three possible forward reading frames. More particularly, the epitope encoding sequences in reading frames 1-3 are c-myc, FLAG, and V5, respectively.

FIG. 13 is a schematic representation of the vector pBLOCK4.0 (SEQ ID NO: 9) for expressing a peptide in a cell, which vector comprises the features of pBLOCK-3.0 (SEQ ID NO: 1) as described in the legend to FIG. 5, except that the gene encoding blasticidin resistance (Bsd) is substituted for a sequence (G418) that encodes a protein conferring resistance to kanamycin in bacteria or resistance to gentamycin in eukaryotic cells.

FIG. 14 is a schematic representation of the vector pRT2 (SEQ ID NO: 10) containing the following features:

(i) a first fluorescent reporter gene cassette comprising the gfp gene encoding green fluorescent protein placed operably under control of a chimeric yeast operable LexA/GAL1 promoter having 8 LexA operator sites, and upstream of the yeast ADHI terminator;

(ii) a second fluorescent reporter gene cassette comprising the cobA gene encoding a red fluorescent protein placed operably under control of a chimeric cl/GAL1 promoter having 3 cl operator sites;

(iii) a wild-type yeast operable selectable marker gene (ADE2) for conferring adenine auxotrophy on cells expressing said gene;

(iv) a selectable marker gene for conferring resistance to the antibiotic kanamycin in bacteria;

(v) a bacterial origin of replication (colE1); and

(vi) a eukaryotic origin of replication (2 μOri).

The present invention clearly extends to the use of the methods and vectors described herein to identify novel drugs, such as, for example, antibiotics or inhibitory agents. In fact, the present invention is particularly useful in drug screening protocols to identify candidate agonists and antagonists of any biological interaction. For example, bacterial expression systems may be used in high through-put screening for novel antibiotics or other inhibitory agents which target specific protein: DNA or protein: protein interactions. The pBLOCK series of vectors described herein are particularly useful in such applications.

### DETAILED DESCRIPTION OF THE PREFERRED EMBODIMENTS

This invention provides a modified reverse two hybrid screen compared to that described in International Patent Application No. PCT/AU99/00018 (WO 99/35282), In particular, whilst the reverse two hybrid screen for peptides that inhibit a target interaction between two proteinaceous binding partners can be performed as described previously, an additional selection is made for peptides that do not inhibit a non-target interaction between one of said proteinaceous binding partners and another binding partner. This means that the bait and prey constructs, and the reporter gene constructs for assaying the target interaction can be any of those described previously. However, to assay the non-target interaction, the host cell must contain an additional “bait” construct and reporter gene construct other than that used to assay the target interaction, for assaying the non-target interaction.

Additionally, it is particularly preferred for dual reporter genes to be operably and separately connected to the target interaction in the host cell, to reduce background. For example, two counter selectable reporter genes can be used for the target interaction to eliminate the potential background of 5-FOA-resistant colonies that arise from mutations in the URA3 reporter gene.

Preferably, the yeast strain is modified to allow the introduction of an additional plasmid expressing a candidate peptide being assayed for inhibitory activity against the target interaction but not the non-target interaction.

The development of new and improved reverse n-hybrid screens that are particularly useful for high-throughput screening of peptide aptamer inhibitors of protein-protein interactions is described below. The detailed description of the preferred embodiments presented herein is for the purposes of exemplification only and should not be construed as placing a limitation on the presently described invention.

### EXAMPLE 1

Generalized Scheme of Improved Reverse Two Hybrid Screen

A generalized scheme for the reverse two hybrid screen of the invention is presented in FIG. 1. In this embodiment, gene constructs for the target interacting partners (designated X and Y in FIG. 1) are prepared, based on the vectors pJG4-5 and pGilda that have been described for use with the lexA-based two hybrid system (Gyuris et al, 1993; Ausubel, 1989). The coding region of the prey, PROTEIN-X, is expressed as an activation domain fusion. The coding region of the target bait, PROTEIN-Y, is expressed as a LexA fusion. In this scheme, a non-target interaction between the prey PROTEIN-X and a non-target bait, PROTEIN-Z (designated Z in FIG. 1) is also assayed. The non-target bait, PROTEIN-Z, is expressed as a cl-repressor fusion from pGMS13; a derivative of pGMS12 (Serebriiskii, 1999) that contains HIS5 in place of the G418 selectable marker gene.

Alternative cl-bait vectors include pGKS8, pGMS12 (Serebriiskii, 1999) or pCIAuri (a derivative of the pAUR112 CEN/ARS vector (Panvera corporation), which contains a GAL1 promoter Cl-repressor and ADH1 terminator in place of the URA3 gene), can also be used. These vectors contain alternative markers allowing selection for Zeocin, G418, or Auriobasidin resistance, respectively.

The reporter genes for the target interaction between PROTEIN-X and PROTEIN-Y consist of two chimeric counter selectable reporter genes URA3 and CYH2, and the green fluorescent protein-encoding gfp gene (Prasher et al., 1992; Chalfie et al., 1994; Inouye and Tsuji, 1994), each comprising one or more LexA operator sequences to which the target bait can bind. It should be noted that the CYH2 selection requires a cycloheximide resistant yeast background containing the cyh2^{R} allele. The reporter genes for the non-target interaction include the single chimeric counter selectable reporter gene LYS2, and the red fluorescent protein-encoding cobA gene (Wildt and Deuschle, 1999), each comprising one or more cl operator sequences to which the non-target bait can bind.

Yeast cells are transformed with gene constructs expressing the prey, target bait, non-target bait, and all reporter gene constructs, and these features are then recombined into single cells by mass mating (Feilotter et al, 1994). The prey, and the target and non-target baits are all expressed from the CEN/ARS-based GAL1 expression vectors, pGilda or pCIAuri. Clearly, this method is not restricted to the bait being expressed from such plasmid vectors, since the expression cassettes could be integrated into the genome without adverse effects.

Vectors marked with TRP1, HIS3, and HIS5, encoding the prey, target bait, and non-target bait are transformed into yeast strain PRT473; a cycloheximide resistant derivative of strain SKY473 having the genotype (MAT-a, cyh2R his3, trp1, lexAop-LEU2, lexAop-CYH2ade2::ZEO^{R}, lexAop-URA3his5::G418R, clop-LYS). The recombinants are screened in parallel for the interacting target and non-target partners.

Because the binding partners of the target interaction are expressed under control of the galactose-inducible GAL1 promoter, it is possible to fine-tune their expression relative to the presence of 5-FOA and cycloheximide in the cell growth media. The minimum level of induction required to cause lethality is determined, as follows: The diploid SKY48/SKY473 strain containing the interacting target bait and prey fusion proteins is tested for growth on a panel of selection media, containing 2% (w/v) raffinose (which is neutral with respect to GAL1 induction), pre-determined concentrations of cycloheximide and 5-FOA, and an amount of galactose in the range 0% (w/v) galactose to 2% (w/v) galactose. The minimum concentration of galactose required for complete lethality of this strain in the presence of 5-FOA and cycloheximide is then determined.

In the absence of any inhibition, the yeast strain is sensitive to the presence of 5-FOA and cycloheximide in growth medium following induction by galactose, due to the interaction between the target bait and prey fusion proteins bound to the LexA operator site(s) of the counter selectable reporter genes URA3 and CYH2. The same interaction makes the yeast cell fluoresce green, due to the expression of the gfp gene. Additionally, interaction between the non-target bait and the prey makes the cell prototrophic for lysine biosynthesis, as a consequence of LYS2 gene expression. Finally, the cell also fluoresces red as a consequence of the interaction between the non-target bait and prey fusion proteins activating cobA reporter gene expression. The phenotype of the cell in the absence of any inhibition is shown in Panel A of FIG. 1.

To screen for specific peptide inhibitors of the target interaction, a further gene construct, in particular any one of the pBLOCK-3 series of vectors shown in FIGS. 5-12, or the pBLOCK-4.0 vector shown in FIG. 13, is modified to express a peptide aptamer. Preferably, a library of peptide aptamers is produced using such vectors. Individual recombinant DNAs of the library are separately introduced into PRT48; a cycloheximide resistant yeast strain derived from strain SKY48 (Serebriiskii, 1999), having the genotype: (MATa, leu2, ade2, ura3, his3, his5 lys2, trp1, cyh2R). The transformants are frozen until required.

The library is introduced into the recombinant PRT 473 strains expressing the binding partners (see above) by mass mating. Diploid yeast cells arising from the mass mating of recombinants derived from strains PRT 473 and PRT 48 are selected on minimal media containing Blasticidin (pBLOCK-3 series of vectors) or G418 (pBLOCK-4.0 vector) and Zeocin, but lacking leucine, tryptophan and histidine, and stored frozen as glycerol stocks.

Screening of the library is then performed by plating the yeast strains, at 10-fold the original library complexity, on minimal media lacking leucine, tryptophan, and histidine, but containing blasticidin (derivatives of the pBLOCK-3.0 series of vectors) or G418 (pBLOCK-4.0 vector derivatives), 5-FOA and/or cycloheximide. Induction of gene expression is achieved by adding the appropriate amount of galactose as determined supra to the growth media.

Panel B of FIG. 1 shows the expected expression profile and selection characteristics of the transformed yeast cells expressing peptide aptamers. Where the inhibitory peptide aptamer prevents interaction between the target bait and prey, no expression of the URA3 or CYH2 counter selectable reporter genes, or the gfp reporter gene, is detected following galactose induction. Accordingly, the yeast strain is made resistant to 5-FOA and cycloheximide, however no longer fluoresces green. On the other hand, cells that do not express an inhibitory peptide with respect to the target interaction will not survive on media containing 5-FOA and/or cycloheximide.

If the inhibitory peptide fails to inhibit the interaction between the prey and the non-target bait, the surviving cells remain prototrophic for lysine biosynthesis, due to the activating interaction between the non-target bait and prey fusion proteins bound to the cl operator sites of the LYS2 reporter gene, which prototrophy can complement an auxotrophic lys2 mutation in yeast. The strain will also continue to express the cobA gene, and, as a consequence, fluoresces red. On the other hand, if the inhibitor peptide also inhibits the non-target interaction, the surviving cells will not grow on media lacking lysine, and will not fluoresce red.

Accordingly, specificity of the inhibition of reporter gene expression is determined by replica plating colonies onto the identical media and media lacking lysine. Colonies that grow in the presence of 5-FOA and cycloheximide, however fail to grow on media lacking lysine express peptide inhibitors that inhibit both the target and non-target interactions. Colonies that grow in the presence of 5-FOA and cycloheximide, and on media lacking lysine express peptide inhibitors that specifically inhibit the target interaction.

### EXAMPLE 2

Reverse Two Hybrid Assay for Peptide Inhibitors of the SCL/LMO2 Interaction

It will be apparent to the skilled worker how to vary the protocol provided in the preceding example, to assay for peptide inhibitors of a particular protein-protein interaction, using the reverse two hybrid screen of the invention. The following modifications are made to the preceding example.

Two parallel reverse two hybrid screens are performed, as shown in FIGS. 2 and 3. The first screen (FIG. 2) identifies peptide inhibitors of a target interaction between the prey, SCL, and a target bait, LMO2, that do not inhibit a non-target interaction between said prey and a non-target bait, E47. The interaction between SCL and E47 has been previously described in Mahajan et al (1996). The second screen (FIG. 3) identifies peptide inhibitors of the same target interaction that do not inhibit another non-target interaction between the prey, SCL, and the non-target bait, LMO1. Accordingly, both non-target interactions can be distinguished from the target interaction. Clones expressing peptide inhibitors of the target interaction alone, or the target interaction and one or both of the non-target interactions can also be identified.

The SCL prey is an existing activation domain/SCL fusion protein, containing the bHLH domain (residues 176-245) of SCL that have been implicated in the interaction of LMO2 (Wadman et al., 1994).

Three counter selectable reporter genes are employed in each two hybrid screen. The first two reporter genes, URA3 and CYH2, are under the control of LexA operators and are therefore dependent on the target interaction. This dependence is because the target bait, LMO2, is expressed as a lexa fusion protein in both screens (FIG. 2 and FIG. 3). As with the previous example, these screens each exploit the toxicity of the URA3 gene product in the presence of the drug 5-fluoro-orotic acid (5-FOA), and the toxicity of the wild-type CYH2 gene product in the presence of the drug cycloheximide. Accordingly, any activation of reporter gene expression arising from the target interaction between SCL and LMO2 is selected against in the presence of the drugs 5-FOA and cycloheximide, as described in the preceding example.

The counter selectable reporter genes for detecting non-target interactions (i.e. SCL/E47 and SCL/LMO1) is the LYS2 gene which is under the control of cl operator sequences (FIG. 2 and FIG. 3). Expression of the LYS2 reporter gene depends on binding of the non-target bait proteins, E47 (FIG. 2) and LMO1 (FIG. 3), which binding is achieved by expressing these non-target baits as fusion proteins with cl operator DNA-binding domains.

Absent any inhibition of SCL and LMO2 binding in either screen, and subsequent inhibition of URA3 and CYH2 reporter gene expression, the yeast strains are sensitive to 5-FOA and cycloheximide following induction by galactose. In contrast, when an inhibitory peptide aptamer prevents interaction between SCL and LMO2, no expression of the URA3 or CYH2 counter selectable reporter genes is detected following galactose induction, and, as a consequence, the yeast strain is made resistant to 5-FOA and cycloheximide. Cells that do not express an inhibitory peptide with respect to the target interaction do not survive on media containing 5-FOA and/or cycloheximide.

The cells of both screens that survive on 5-FOA and cycloheximide are assayed further for their lysine requirements, as described in the preceding example. Absent inhibition of the SCL/E47 interaction (FIG. 2) or the SCL/LMO1 interaction (FIG. 3), the cells are prototrophic for lysine. Accordingly, those cells that survive of 5-FOA and cycloheximide are also prototrophic for lysine biosynthesis, the interaction between SCL and E47 (FIG. 2), or between SCL and LMO1 (FIG. 3), is not inhibited, and the inhibitory peptide is specific for the target interaction between SCL and LMO2. On the other hand, in either of the parallel screens, the yeast strains that grow on 5-FOA and cycloheximide, but do not grow on media lacking lysine, express an inhibitory peptide that also inhibits the particular non-target interaction in question (i.e. the SCL/E47 interaction or the SCL/LMO1 interaction). Accordingly, it is possible to determine those peptide aptamers that inhibit one or two or three of the interactions assayed that involve SCL. Those skilled in the art will readily be able to assay additional interactions involving the SCL protein, by performing additional screens, each having a different non-target bait, in the manner described herein.

### EXAMPLE 3

Reverse Three Hybrid Screen for Peptide Inhibitors of an RNA/Protein Interaction

Reverse three hybrid screens are described in International Patent Application No. PCT/AU99/00018 (WO 99/35282).

In the reverse three hybrid method described herein, a constant bait construct is required that expresses a DNA binding domain, such as, for example, GAL4 or LexA, as an in-frame fusion with an RNA-binding protein, such as, for example, the coat protein MS2 (Invitrogen) which contains an RNA-binding cleft. Preferably, this construct is integrated (Invitrogen). More preferably, the construct is contained on a yeast plasmid containing the 2 micron or CENIARS origin of replication.

Additionally, a second RNA-bait construct is required that expresses a hybrid RNA comprising an RNA target and the ligand for the RNA-binding protein of the constant bait. For example, a hybrid RNA-encoding gene comprising MS2 RNA sequences can be used to bind to an MS2 coat protein domain of the constant bait. Examples of such RNA-bait constructs include the vectors pRH3′ and pRH5′ (Invitrogen). 30

Additionally, a specific prey construct is also required, expressing the protein that interacts with the target RNA of interest, fused to a transcriptional activation domain. This prey construct is of a standard form, as for two hybrid screening, such as, for example, pJG4-5, or pYESTrp2, or pACT2. Preferably, the prey construct is pJG4-5. More preferably, the prey construct is pYESTrp2.

In other respects, the principles for three performing reverse hybrid screens are essentially as described herein for reverse two hybrid screens.

To screen for candidate peptide inhibitors of the RNA/protein interaction, a peptide library expressed from a vector such as the pBLOCK-3 series of vectors described herein (FIGS. 5-12) or pBLOCK-4.0 (FIG. 13) can be used. The recombinant library is transformed into a yeast strain expressing the constant bait protein, the RNA bait and the prey protein in the manner described for the preceding examples.

A non-limiting example of the reverse three hybrid screen of the invention is present in FIG. 4. In this screen, inhibitors of the target interaction between the TAT protein and RNA encoding TAR protein are identified, and selected preferentially over peptides that only bind to non-target interactions involving TAT protein, RNA encoding TAR protein, MS2 RNA, or MS2 protein.

The prey for both the target interaction (and any non-target interaction, not indicated) is an RNA binding protein (TAT) expressed as a fusion protein with an activator domain as described in the preceding examples. The RNA bait is a hybrid MS2/TAR RNA molecule comprising at least the RNA-binding cleft of the MS2 coat protein-encoding RNA and TAR-encoding RNA that binds to TAT. The constant bait is a fusion protein between MS2 protein and the DNA binding domain of the LexA protein. The reporter genes for the target interaction consist of chimeric URA3 and CYH2 genes comprising one or more LexA operator sequences to which the bait can bind, as described in the preceding examples.

The non-target bait comprises a fusion protein between any other protein to which the MS2/TAR hybrid RNA can bind, (either through the MS2-binding nucleotide sequence moiety or the TAT-binding sequence) and the cl repressor protein. The reporter gene for the non-target interaction consists of a chimeric LYS2 gene comprising one or more cl operator sequences to which the non-target bait can bind, as described in the preceding examples.

Expression of the URA3 and CYH2 counter selectable reporter genes requires the formation of a complex between the LexA operator and the LexA-MS2 fusion protein and the MS2-TAR hybrid RNA and the TAT-activator domain fusion protein. Accordingly, this interaction makes the cells susceptible to 5-FOA and cycloheximide following galactose induction of expression of the LexA-MS2 fusion protein and/or the TAT-activator domain fusion protein. Similarly, expression of the LYS2 gene when a non-target interaction occurs in the cell makes the cell prototrophic for lysine. Accordingly, absent any inhibition of formation of the above complex, the yeast strain is sensitive to 5-FOA and cycloheximide, and prototrophic for lysine biosynthesis.

Inhibition of the target interaction between TAT protein and TAR-encoding RNA, will confer resistance to 5-FOA and cycloheximide on the cell. Such cells express a peptide that inhibits the target interaction. The peptide also inhibits a non-target interaction that normally activates LYS2 reporter gene expression, when the cells require lysine for their growth. On the other hand, the peptide is specific for the target interaction when thew cells remain prototrophic for lysine.

### EXAMPLE 4

Construction of New Vectors for Expressing Candidate Peptide Inhibitors.

We have prepared a series of vectors suitable for expressing peptide aptamers to be screened in the reverse n-hybrid assays described herein. The pBLOCK-3 series of vectors described herein comprise the following vectors: pBLOCK-3.0 (FIG. 5 and SEQ ID NO: 1); pBLOCK-3.2 (FIG. 6 and SEQ ID NO: 2); pBLOCK-3.4 (FIG. 7 and SEQ ID NO: 3); pBLOCK-3.6 (FIG. 8 and SEQ ID NO: 4); pBLOCK-3.8 (FIG. 9 and SEQ ID NO: 5); pBLOCK-3.9 (FIG. 10 and SEQ ID NO: 6); pBLOCK-3.10 (FIG. 11 and SEQ ID NO: 7); and pBLOCK-3.11 (FIG. 12 and SEQ ID NO: 8).

These vectors contain many features described for the vectors pBLOCK-1 and pBLOCK-2 as described in International Patent Application No. PCT/AU99/00018 (WO 99/35282), including the tandem promoter features for expressing the candidate peptide in both prokaryotic and eukaryotic cells; and the presence of both E. coli and yeast replication origins to facilitate their use in both bacterial and yeast cells. As with pBLOCK-1 and pBLOCK-2, the constitutive strong ADH promoter drives expression of the candidate peptides in yeast, to allow high level expression that is independent of the carbon source. The SV40 nuclear localization sequence to direct the candidate peptide to the nucleus is also present. As with pBLOCK-1 and pBLOCK-2, the peptide is expressed as a fusion with an epitope tag (V5) to facilitate immune precipitation or Western blotting of the expressed candidate peptide.

Additionally, the vectors described herein are small (approximately 6 kilobase pairs), to facilitate complex library construction.

All pBLOCK-3 series of vectors contain the Bsd gene conferring resistance to the drug Blasticidin in yeast and bacterial cells. The gene is flanked by both bacterial and yeast transcriptional control sequences enabling the rapid, specific rescue of library plasmids encoding candidate peptide inhibitors by transformation of E. coli and selection on rich media containing Blasticidin.

Particular derivatives of pBLOCK-3.0 incorporate a range of additional features, as described in the corresponding figure legends:

(i) A single chain antibody gene (SCAG) operably under control of the SV40 promoter (vector pBLOCK-3.8) allows detection of transfected eukaryotic cells using antibodies.

(ii) Site-specific recombinase sites (e.g. f, att, loxP, res), flanking sequences encoding the candidate peptide in vectors pBLOCK-3.6, pBLOCK-3.8 and pBLOCK-3.9, facilitate rapid transfer of inserts from this vector into another vector via site specific recombination mediated by the corresponding recombinase enzymes (e.g. Flp recombinase, lambda integrase, Cre recombinase, and transposon resolvase, respectively).

(iii) positive selection genes encoding toxic products, in particular the ccdB gene operably under the control of the lac promoter (vectors pBLOCK-3.4, pBLOCK-3.6, pBLOCK-3.8, pBLOCK-3.9, pBLOCK-3.10, and pBLOCK-3.11) facilitates cloning and/or insert transfer by recombination.

Furthermore, the vector pBLOCK-4.0 (FIG. 13 and SEQ ID NO: 9) is identical to the vector pBLOCK-3.4 except that the Bsd gene has been substituted with the G418 gene that confers kanamycin resistance in bacteria and gentamycin resistance in eukaryotic cells.

The features of each plasmid are summarized in Table 1. Additional salient or unique features of each of these vectors will be apparent from the Sequence Listing and the drawings.

It is clear to those skilled in the art that the utility of the shuttle vectors such as those of the pBLOCK family are not restricted to reverse two hybrid screening. They are also useful for the cloning of a range of DNA molecules for a variety of purposes well known to those skilled in the art, including cDNA, genomic, PCR and oligonucleotide cloning (Ausubel et al, 1989). Minor modifications to the sequences of these pBLOCK vectors by means of small deletions, insertions and/or transitions or transversions, would not have a significant effect on the function of these vectors or to constitute an inventive step. Accordingly, all such equivalent vectors (i.e. variants or derivatives) are encompassed by the present invention.

TABLE 1

| FEATURE | BLOCK-3.0 | BLOCK 3.2 | BLOCK 3.4 | BLOCK 3.6 | BLOCK 3.8 | BLOCK 3.9 | BLOCK 3.10 | BLOCK 3.11 | BLOCK 4.0 |
|---|---|---|---|---|---|---|---|---|---|
| ORIGINS OF | | | | | | | | | |
| REPL- | | | | | | | | | |
| ICATION | | | | | | | | | |
| 2 μm ori | ✓ | ✓ | ✓ | ✓ | ✓ | ✓ | ✓ | ✓ | ✓ |
| pMB1 ori | ✓ | ✓ | ✓ | ✓ | ✓ | ✓ | ✓ | ✓ | ✓ |
| SELECTABLE | | | | | | | | | |
| MARKER | | | | | | | | | |
| GENE | | | | | | | | | |
| bsd gene | ✓ | ✓ | ✓ | ✓ | ✓ | ✓ | ✓ | ✓ | |
| g418 gene | | | | | | | | ✓ | |
| amp^{r} gene | ✓ | | ✓ | ✓ | ✓ | ✓ | ✓ | ✓ | ✓ |
| TANDEM | ✓ | ✓ | ✓ | ✓ | ✓ | ✓ | ✓ | ✓ | ✓ |
| PROMOTERS | | | | | | | | | |
| CMV/ADH/T7 | ✓ | ✓ | ✓ | ✓ | ✓ | ✓ | ✓ | ✓ | ✓ |
| SV40/TEF/ | ✓ | ✓ | ✓ | ✓ | ✓ | ✓ | ✓ | ✓ | ✓ |
| EM7 | | | | | | | | | |
| TOXIN | | | | | | | | | |
| GENES | | | | | | | | | |
| lac-ccdB | | ✓ | ✓ | ✓ | ✓ | ✓ | ✓ | ✓ | ✓ |
| EPITOPE | | | | | | | | | |
| TAGS | | | | | | | | | |
| V5 epitope | ✓ | ✓ | ✓ | ✓ | ✓ | ✓ | ✓ | ✓ | ✓ |
| C-myc epitope | | | | | | | | ✓ | |
| RE- | | | | | | | | | |
| COMBINASE | | | | | | | | | |
| SITES | | | | | | | | | |
| attL1/attL2 | | ✓ | | ✓ | ✓ | | | | |
| LoxP sites | | | | | | ✓ | | | |
| SV40 NLS | ✓ | ✓ | ✓ | ✓ | ✓ | ✓ | ✓ | ✓ | ✓ |
| ADHTT | ✓ | ✓ | ✓ | ✓ | ✓ | ✓ | ✓ | ✓ | ✓ |
| IRES | | | | | ✓ | | | | |
| SCAG | | | | | ✓ | | | | |

### EXAMPLE 5

Construction of Random Peptide Aptamer Libraries

Conventional two hybrid libraries are fused to a transcriptional activation domain and are not suitable for reverse n-hybrid screens, because they do not allow the cloning of various types of peptide inhibitors wherein some members of the library activate transcription and thereby evade the screen, regardless of whether or not they block the interaction under target. Moreover, an additional selectable marker in the yeast vector, and an appropriate strain for its selection, are preferred.

Existing rational design approaches attempt to model candidate therapeutic peptides based on homologies in the databases to natural inhibitory peptides (e.g. Tiozzo et al., 1998). Such existing approaches focus on peptides/polypeptides which have previously been identified from their natural source due to their inhibitory properties. In contrast the screens described herein, empirically determine the most suitable peptides from a wide array of candidate peptides encoded in a genomic expression library. The presence of clones in all reading frames, using a vector such as pBLOCK-3.11, also allows the simultaneous screening of random peptides expressed in reading frames which do not occur in nature, together with a variety of natural peptide domains cloned in the appropriate reading frame.

Preferably, the Trx-presented random peptide libraries for the reverse two hybrid screen. The preparation of the random inserts constrained within the Trx loop is identical to that described by Colas (1996), except that the preferred vector is optimized for high through-put applications, such as, for example, a pBLOCK vector described herein.

To improve the proportion of appropriately folded domains in the repertoire available for screening, peptide libraries are constructed in the pBLOCK series of vectors described in the preceding example, using randomly selected sequences derived from a phylogenetically diverse compact genomes. To maximize the diversity of the pool, the relative concentration of DNA in the pool from larger genomes is increased in proportion to the total haploid genome size. Such libraries are constructed according to standard methodology (Ausubel, 1989) using the highest efficiency available competent cells, for example XL10-Gold (Stratagene), to ensure complexities greater than 10⁷ independent clones.

To maximize the diversity of the pool of potential inhibitory peptide aptamers, whilst taking advantage of the structural information present in naturally-evolved gene sequences, libraries of inhibitory peptides are preferably constructed from natural genome sources. This approach attempts to accelerate the evolutionary process, by artificially combining domains from different genomes which is unlikely to co-evolve. To achieve this end, genomic expression libraries from evolutionarily diverse organisms are produced, such as, for example, Fugu rubripes (Elgar, 1996a, 1996b), Caenorhabditis elegans (Plasterk, 1999), and Saccharomyces cerevisiae. Alternatively, libraries are constructed using pooled genomic DNA from these organisms, or microorganisms that have been characterized at the genetic level. While the potential diversity achieved by such an approach is theoretically less than that achieved by cloning and expressing DNA purified directly from the environment (Short, 1997), it offers several distinct advantages.

For example, true diversity and bias of the library is more easily approximated, allowing the operator to maximize domain diversity and minimize bias towards the genomes of dominant species.

Additionally, artificial pooling of DNA derived from distinct known organisms allows unique opportunities to survey diverse genomes. For example, the genomes of certain archaebacteria are simultaneously screened with those of obligate parasites, such as Mycoplasma, and/or diverse gram positive and/or gram negative organisms.

Additionally, the diversity of such domain libraries is increased further by mutation. For example, the plasmid library is amplified in bacterial strains that are deficient in mismatch repair (e.g. strains containing the mutS mutation), resulting in the generation of mutations.

### EXAMPLE 6

LexA-Responsive Counter-Selectable CYH2 Reporter Gene

A panel of constructs have been generated containing up to eight LexA operator DNA binding domains (DBD) embedded in a GAL10/GAL1 basal promoter, configured to drive expression of a CYH2 reporter gene in yeast. Immediately downstream from CYH2, a Zeocin resistance gene (ZEO) is inserted in the reverse orientation. Both yeast and bacterial promoters/transcriptional terminators direct expression of the ZEO gene. The constructs were amplified by PCR, using primers containing 3′ tags homologous to the ADE2 gene, thereby facilitating their targeted integration to gene constructs having the ADE2 gene. Stable integrants were selected for Zeocin resistance and screened for red pigmentation (adenosine auxotrophy).

### EXAMPLE 7

LexA-Responsive Counter-Selectable URA3 Reporter.

A panel of constructs have been generated containing up to eight LexA operator DNA binding domains (DBD) embedded in a GAL 10/GAL1 basal promoter, configured to drive expression of a URA3 reporter gene in yeast. Immediately upstream from the GAL10/1 promoter, the G418 gene is inserted. Expression of G418 is controlled by the TEF1 promoter, which functions in both yeast and bacteria. The constructs were amplified using PCR primers containing 3′ tags homologous to the HIS5 gene and transformed into yeast. Stable integrants were selected for G418 resistance and screened for histidine auxotrophy.

There is a potential for inadvertent cell death upon counter selection, caused by non-specific activation of the basal promoters fused to reporter genes, via interaction with library products expressed from the pBLOCK3 series of vectors. The impact of such interactions is limited by employing different basal promoter-reporter gene fusions in the same two-hybrid system, since the likelihood of the same expressed library product activating distinct promoters is very low. Hence, the present invention contemplates the use of alternate basal promoters in place of GAL1/10, to drive the URA3 counter-selectable reporter. Accordingly, LexAop-URA3 reporter genes, containing either SPO13 (Vidal et al, 1996a,b), or CUP1-URA3 fusion genes are constructed. Embedded in these basal promoters are up to eight LexA operator sequences. Constructs are integrated in the SKY473 genome at the HIS5 locus via a double crossover recombination event (Huang and Schreiber, 1997).

### EXAMPLE 8

The Dual Fluorescent Reporter Gene Vector pRT2

A novel dual fluorescent reporter gene vector was produced. This vector, designated pRT2 (FIG. 14 and SEQ ID NO: 10) is derived from pRG64, which contains a GALIO/GALL basal promoter with three cl binding sites operably connected to expression of the β-glucuronidase (Gluc) reporter gene.

To produce the intermediate vector pRT1, plasmid pRG64 was digested with HindIII, and the ends filled-in and re-ligated to generate a unique Nhe I site immediately downstream of the URA3 counter selectable reporter gene. A fragment of 2,276 bp in length, containing a minimal complementing region of the yeast ADE2 genomic locus, was amplified using PCR, and inserted at this Nhe I site. The URA3 gene was deleted using NotI and Bsal, and a DNA fragment containing a GAL10/GAL1 basal promoter having six cl binding sites operably connected to expression of the CobA gene, was inserted into the same sites.

To generate pRT2 (FIG. 14), plasmid pRT1 was cut with BamHI, immediately 5′ of the GAL-Gluc reporter, and the ends filled-in, and the blunt-ended fragment re-ligated, to generate a ClaI site. The entire GAL-Gluc reporter was excised by digestion with Clal, and a 1800 bp fragment, containing a GAL10/GAL1 basal promoter having eight LexA binding sites operably connected to the expression of the CobA gene, was inserted into the Clal site.

The vector contains dual fluorescent reporters allowing real-time assessment of reporter gene activation without the need for substrates. The vector also allows assessment of reporter gene activation via baits fused to LexA and/or cl DNA Binding Domains (DBD's) Incorporating both fluorescent reporters, each dependent on distinct DBD's, in the one vector, eliminates confounding effects associated with variability in reporter gene expression due to variation in plasmid copy number observed when the reporters are expressed on different plasmids

The dual fluorescent reporter construct is transformed into yeast strains, that are auxotrophic for Adenine (ADE2) thereby selecting for maintenance of the vector. Transformation of the aforementioned strains with prey and baits, the latter fused to LexA and/or cl DBD's, allows for activation of the fluorescent reporters. In the case of a forward two-hybrid yeast screen, successful interaction between baits and preys would result in activation of the relevant fluorescent reporter, which is measured by fluorescent-based detection systems familiar to those skilled in the art. The converse is true for reverse hybrid screening where one is looking for down-regulation of reporter gene expression resulting from the inhibition of interactions between bait and prey proteins.

### EXAMPLE 9

The Dual Colorimetric Reporter Gene Vector pRT3

The dual reporter plasmid pRT3 is derived from pDR8, which contains a GAL10/GAL1 basal promoter having three cl binding sites directing expression of the Gluc reporter gene, and a GAL10/GAL1 basal promoter having eight LexA binding sites directing expression of a LacZ reporter gene.

The URA3 selectable marker contained in pDR8 is exchanged for the ADE2 gene by digesting the plasmid with HindIII, end-filling and re-ligating the blunt-ended fragment, to generate a unique NheI site. A DNA fragment containing the URA3 gene is excised following digestion with NheI and BsaI, and a 2,276 bp PCR fragment, containing a minimal complementing region of the yeast ADE2 genomic locus, is inserted at the same sites, generating pRT3.

### EXAMPLE 10

Host Strains

The parent host strain SKY473 contains the cl-responsive LYS2 reporter gene. It has the genotype (MAT-a, his3, trp1, lexAop-Leu2, clop-LYS2).

Particular constructs described in the preceding examples are transformed into a cycloheximide resistant derivative of SKY 473 (MAT-a, his3, trp1, lexAop-Leu2, clop-LYS2, cyh2R), generating the strain PRT 473 (MAT-a, cyh2^{R}his3, trp1, lexAop-LEU2, lexAop-CYH2ade2::ZEOR, lexAop-URA3his5::G418 R, clop-LYS). Using the same starting strain, different gene constructs produce strain PRT 475 (MAT-a, cyh2^{R} his3, trp1, ade2, lexAop-GAL1-LEU2, lexAop-GAL1/10-CYH2, lexAop-CUP1-URA3, and clop-GAL1-LYS2).

Strain PRT 474 differs from strain PRT 475 only by the choice of repressed promoter, which drives URA3 expression. The CUP1 promoter has the advantage of being copper inducible, thereby allowing titration of repression, by altering the concentration of copper in the medium. Methods for such titration of the degree of repression of the CUP1 promoter are known to those skilled in the art (Schneider, 1991).

### EXAMPLE 11

Adjusting Selection Threshold by Titration of Galactose.

The reverse two hybrid screening system of the present invention allows the primary adjustment of stringency threshold for any interacting partners expressed from the GALL promoter. Since both the bait and prey interactors are expressed under the control of the GALL promoter, the expression of these proteins in the cell is varied by modifying the level of galactose in the media between no galactose and 2% (w/v) galactose. The concentration of the neutral sugar, raffinose, is maintained at a constant 2%(w/v) concentration.

The galactose induction system facilitates determination of the minimum concentration of binding partner expression required for a counter selectable reporter gene to be expressed at a level that causes cell death, at a predetermined concentration of 5-FOA or cycloheximide. This overcomes the problem of auto-activation of some bait constructs, which causes cell death in the absence of an interacting partner.

The means for determining an appropriate concentration of galactose to induce expression of binding partners is described in Example 1 supra.

### EXAMPLE 12

Identifying High Affinity Peptide Inhibitors Using Surface Plasmon Resonance

Surface plasmon resonance has rapidly become the benchmark technique for the physical measurement of biomolecular interactions. The instrument makes use of an evanescent wave phenomenon to produce a detectable signal from very subtle refractive index changes on the surface of a microchip, such as, for example, a biosensor chip. Accordingly, the binding, or dissociation, of even a few protein molecules to a partner protein molecule bound to the microchip surface is measured in real-time. This facilitates the derivation of real-time association and dissociation constants. Thus, plasmon resonance is ideally suited to study interactions of proteins identified in reverse n-hybrid screens.

Furthermore, the sensitivity of surface plasmon resonance instrumentation (e.g. BIAcore2000, Pharmacia), facilitates the identification of components in crude cell extracts that compete with the target interaction, assayed on a microchip surface. For example, SCL, purified as a chitin binding protein fusion using the vector pTYB1 (New England Biolabs) is used as starting material. In an adaptation of the screen described in Example 2, purified SCL is covalently coupled to the microchip surface, and purified LMO2, DRG, or E47, expressed as fusion proteins with glutathione S-transferase (GST) is passed over the chip surface, and the binding is monitored using the BIAcore2000. Yeast extracts that express peptide aptamers, or a vector control Trx polypeptide, are then passed across the chip, and the ability of each aptamer to inhibit the association of cognate partners with SCL is determined by a change in refractive index that is measured by the BIAcore2000. The change in refractive index is measured when the peptide inhibitor binds to SCL or to its partner protein or to both proteins. Since the signal produced is proportional to the size of the interacting protein, the difference between the binding of the cognate partners of SCL and the much smaller artificial aptamer, should it interact directly with SCL, can be detected.

The reverse two hybrid assay is repeated, using unrelated pairs of proteins to ensure specificity of the interaction.

Peptide aptamers that inhibit interactions with highest affinity and specificity are immunopurified via their Trx domain or other epitope domain, and tested for direct interactions with the binding partners. Their dissociation constants, and sequence are then determined.

The inhibitory effect of peptide aptamers is initially tested by expressing them constrained within the Trx polypeptide loop in cell lines. The 20mer peptides are synthesized as cyclic peptides fused to the Dropsophila penetratin motif. Such cyclization is achieved by including flanking cysteine residues in the synthetic peptides. This maintains high affinity, by conformationally constraining the peptides (Giebel et al., 1995). Fusion to the penetratin motif facilitates administration of the peptides to the medium of a cell, or the extracellular space. This approach has yielded several biologically active peptide inhibitors.

### EXAMPLE 13

Preliminary Ex-Vivo Validation of Drug Screening Approach

Transfection assays are used to establish whether expression of peptides that inhibit the SCL/LMOS interaction in vitro also inhibit cellular proliferation in a dominant negative manner. Peptides are synthesized in a conformationally constrained form as fusions with the Drosophila ‘penetrating’ targeting sequence, as described in the preceding example. Alternatively, or in addition, the peptides are expressed as a fusion with the VP22 protein of Herpes Simplex Virus Type-1 (Phelan et al., 1998). These cyclic peptides are subsequently administered directly to leukemia cells in culture to determine their ability to inhibit growth or proliferation of the cells.

In the embodiment described herein, the following methods are employed. Leukemia cells are grown in RPMI including 10% FCS, and transfected with plasmids expressing candidate peptide inhibitors by electroporation according to standard protocols. Transfected cells are purified via the single chain antibody to the hapten phOx expressed by the vector pHOOK-2 (Invitrogen) using magnetic beads. Purified transfectants are outgrown in the presence of G418. Standard methods to determine growth by uptake of ³H thymidine from media and clonogenic colony formation assays are routinely used. Enumeration of live and dead cells is performed using a set of probes from Molecular Probes (#L03224).

Expression of peptide inhibitors in cell lines derived from leukemia patients: A model for inhibition of leukemia cell growth.

Plasmids from the pBLOCK vector series described herein allow the direct bioassay of candidate blocking 20 peptides in mammalian cells. Clones are rescued from yeast cultures by the transformation of bacteria such as E. coli using yeast lysates and selection for antibiotic resistance. Selection of the pBLOCK plasmid amongst other plasmids present in the yeast cell is facilitated by ensuring that the antibiotic resistance marker is unique to that plasmid.

Alternatively, promising high affinity peptide inhibitors are cloned in the context of the thioredoxin loop into the mammalian expression vector pHOOK-2 (Invitrogen) under the control of the CMV promoter. This vector also contains a mammalian selectable marker (NEO) and constitutively expresses a single chain antibody to the hapten phOx. Alternatively, where the recipient plasmid contains appropriate flanking sequences for site-specific recombination, the plasmids (eg. pBLOCK-3.2, pBLOCK-3.6 and pBLOCK-3.8) allow the rapid transfer of inserts from the library into the desired expression plasmid using a recombinase reaction. In one preferred embodiment, this step is eliminated in vectors of the pBLOCK family which already contain genes encoding single chain antibodies for physical selection via their cognate epitopes/haptens. Preferably, the peptide inhibitor-expressing constructs are transfected into the following cell lines by lipofection or electroporation:

(i) Erythroleukemic K562 cells that express LMO2, DRG, E47 and SCL;

(ii) the cell line PER-255 (Kees et al., 1989) that expresses DRG, E47, and SCL;

(iii) the cell line PER-117 (Kees et al., 1987) that expresses SCL, DRG and E47; and

(iv) the cell line PER-550 (Kees et al, unpublished) that expresses LMO2, SCL, E47 and DRG.

As a control, use of the T-cell line MOLT-4, or the B-cell line Raji, which do not express SCL, but express both DRG and E47 (Green et al., 1991a,b), is contemplated.

Additionally, the expression vector alone is transfected into each cell line supra as a negative control.

Following transfection and growth for 24 hours, the transfected cell population is purified using magnetic beads coated with the phOx-coated hapten. Equal numbers of selected and control cells are cultured and grown in media containing G418. Cell numbers are measured after 24, 48, and 72 hours. Proliferation is determined by measuring ³H-thymidine uptake, and performing clonogenic colony formation assays, using standard procedures. Viability is assessed by enumerating live and dead cells using probes that are amendable to detection by flow cytometry or FACS.

Introduction of Peptides into Leukemia Cell Lines

The peptides used in this experiment were previously selected in vitro for high affinity inhibition of the SCL/LMO2 interaction (Example 2). Inhibitory peptides, and control “scrambled” peptides of equivalent length, are synthesized as fusions with the Drosophila penetratin sequence, and having oxidizable flanking cysteine residues to facilitate their cyclization.

Peptide inhibition of SCL-dependent growth and target gene activation is determined by adding the interacting peptides (and a non-interacting control) directly to cultures of K562, PER-255, PER-550, PER-117, MOLT-4 and Raji cells, and monitoring the effects over 72 hours as described above. Peptides that inhibit interactions in vivo block SCL-dependent growth. In contrast, the control peptide, or peptides having an affinity for a non-expressed partner protein, have no effect on SCL-dependent growth.

### EXAMPLE 14

Design of Peptides That Inhibit Oncoprotein Interactions.

Consensus alignments of sequence information derived from promising peptide inhibitors of the SCULMO2 interaction assist in the elucidation of potential naturally-occurring binding partners for a nuclear oncoprotein. In particular, the aligned sequences define consensus motifs for high affinity binding partners.

In one approach, cDNA libraries constructed from T-ALL cell lines supra are screened using oligonucleotides that encode consensus interaction motif(s), to identify a cDNA encoding a naturally-occurring oncoprotein having the consensus interaction motif(s).

Alternatively, libraries constructed from the pooled genomes of sequenced organisms may be screened and the sequences of the positive hybridizing clones aligned. Whilst the alignment of sequences derived from a screen reveals consensus motifs, other potential related motifs are excluded if they are not identified from any single genome in which they theoretically occur, despite exhaustive screening at a complexity which would be predicted to cover all of the potential domains encoded by the genome/s. Accordingly, the aligned sequences from the screen of pooled genomes are useful for designing optimal peptides that mimic the consensus motifs identified in the biological screen, while lacking alternative residues of structurally related peptides that were included in the exhaustive screen of a single genome.

### EXAMPLE 15

### Peptide Synthesis

38-mer peptides are designed in the form of Cys(Penetratin 16-mer)(interacting 20-mer)Cys. The sequence of the penetrating motif is provided by Derossi (1994). The peptides (synthesized by Chiron Mimotopes, Australia) are cyclized by oxidation of the flanking cysteine residues using 10 mM K3Fe(CN)6 at pH 8.4 and purified by reverse-phase HPLC according to Koivunen (1994).

### REFERENCES

1. Akiyama, et al. (1989) Proc. Natl Acad. Sci (USA) 86:3949-3952.

2. Amann and Brosius (1985) Gene 40:183.

3. Ausubel, F. M., Brent, R., Kingston, R E, Moore, D. D., Seidman, J. G., Smith, J. A., and Struhl, K. (1987-1997) In: Current Protocols in Molecular Biology. Wiley Interscience (ISBN 047150338).

4. Chalfie, M. et al (1994) Science 263:802-805.

5. Colas, P. et al. (1996) Nature 380:548.

6. Condorelli, G. L. et al. (1996) Cancer Research 56:5113.

7. Cormack, B. et al (1996) Gene (in press).

8. Derossi, D. et al. (1994) J. Biol. Chem. 269:10444.

9. Elgar, G. (1996a). Human Mol. Genet. 5:1437-42.

10. Elgar, G., et al. (1996b). Trends in Genetics 12(4): 145-50.

11. Fahraeus, E., et al. (1996) Current Biology 6: 84-91.

12. Feilotter, et al. (1994). Nucl. Acids Res. 22 (8): 1502-3.

13. Fijalowska, I. J., and Schaaper, R. M. (1995) J. Bacteriol. 177:5979-5986.

14. Finley, Jr, R. L., et al. (1994) Proc. Natl. Acad. Sci. (USA) 91:12980.

15. Frick, et al. (1995) J. Biol. Chem. 270: 24086-24091.

16. Giebel, L. B. et al., (1995) Biochemistry 34:15430.

17. Golemis, E., and Brent, R. (1992) Mol. Biol. 12: 3006-3014.

18. Green, A. R., DeLuca, E., and Begley, C. G. (1991b). EMBO J. 10: 4153-4158.

19. Green, A. R. et al., (1991a) Oncogene 6:475.

20. Gyuris, J. E. et al. (1993) Cell 75:791-803.

21. Haas, J. et al (1996) Curr. Biol. 6:315-324.

22. Hsu, H. L. et al. (1991) Mol. Cell Biol. 11:3037.

23. Huang, J. and Schreiber, S L (1997). Proc. Natl. Acad. Sci., 94: 13396-13401.

24. Inouye, S., and Tsuji, F. I. (1994) FEBS Letts 341:277-280.

25. Kees, U. R., et al. (1987). Leukemia Res. 11(5): 489-498.

26. Kees, U. R., et al. (1989). Blood 74(1): 369-373.

27. Kioveunen, E. et al. (1994) J. Cell Biol. 124:373.

28. Kozak, M. (1987) Nucleic Acids Res. 15: 8125-8148.

29. Larson, R. C. et al., (1996) EMBO J. 15 (5):1021.

30. Lu, T-W., and Cillo (1995) J. Biol. Chem. 270:23582-23588.

31. Mahajan, M. A. et al. (1996) Oncogene 12:2343.

32. Maki and Sekiguchi (1992) Nature 355: 273-275.

33. Marcello, A. et al. (1994) Proc. Natl. Acad. Sci. USA 91:8994.

34. Matz, M V et al., (1999) Nature Biotechnology 17: 969-973.

35. Miller, J. H. (1992) A Short Course in Bacterial Genetics. Cold Springs Harbor Laboratory Press, Cold Spring Harbor, N.Y.

36. Miller, J. H., and Michaels, M. (1996) Gene 179:129-132.

37. Moriya and Gollman (1992) Mol. Gen. Genetics 239: 72-76.

38. Osada, et al. (1995) Proc. Natl. Acad. Sci. USA 92:9585.

39. Ow, D. W.; et al. (1986) Science 234:856-859.

40. Phelan, et al (1998). Nature Biotechnology 16(5): 440-443.

41. Plasterk, R. H. A. (1999). Nature genetics 21: 63-64.

42. Prasher, D. C. et al (1992) Gene 111:229-233

43. Rouquet, N et al., (1996) Current Biology 6: 1192-1195

44. Sambrook, J.; Fritsch, E. F.; and Maniatis, T. (1989) Molecular cloning. A Laboratory Manual. Second Edition. Cold Spring Harbor Laboratory, Cold Spring Harbor, N.Y.

45. Schaaper, R. M., and Comacchio, R. (1992) J. Bacteriol. 174: 1974-1982.

46. Schneider, J. a. G., L. (1991). In: Vectors for expression of cloned genes in yeast: Regulation Overproduction and underproduction, Vol.194, (C. Guthrie, Ed) San Diego: Academic Press.

47. Serebriiskii etal (1999) J. Biol. Chem. 274(24):17080-17087.

48. Shimatake and Rosenberg (1981) Nature 292:128.

49. Short, J. (1997). Nature Biotechnology 15: 1322-1323.

50. Sikorski, R. S. et al. (1989) Genetics. 12219-27.

51. Slupska, M.M., et al (1996) Proc. Natl. Acad. Sci. (USA) 93: 4380-4385.

52. Studier and Moffat (1986) J. Mol. Biol. 189:113.

53. Tajiri, et al. (1995) Mutation Res. 336: 257-267.

54. Thompson, J. F.; Hayes, L. S.; and Lloyd, D. B. (1991) Gene 103:171-177.

55. Tiozzo, E., et al. (1998). Biochem. Biophys. Res. Comm. 249: 202-206.

56. Vidal, M. et al. (1996b) Proc. Natl. Acad. Sci. USA 93:10315.

57. Vidal, M., P. et al. (1996a) Proc. Natl. Acad. Sci. USA 93:10321.

58. Wadman, I., J. et al. (1994) EMBO J 13 (20):4831.

59. Warbrick, E., et al., (1996) Current Biology 5:275.

60. Wildt, S and Deuschle, U (1999) Nature Biotechnology 17: 1175-1178

61. Young, K., et al. (1998). Nature Biotechnology 16:946-950.

What is claimed is:

1. A method of identifying a peptide that partially or completely inhibits a target interaction between two or more binding partners in a host cell but does not inhibit a non-target interaction between some but not all of said binding partners, said method comprising:
(i) expressing in a cellular host: (a) the binding partners of said target interaction such that they operably control the expression of one or more reporter genes in said cellular host, wherein said expression is partially or completely inhibited by disruption of said target interaction; (b) the binding partners of said non-target interaction such that they operably control the expression of one or more reporter genes in said cellular host, wherein said expression is partially or completely inhibited by disruption of said non-target interaction and wherein said reporter gene is distinct from the reporter gene(s) expressed under control of the target interaction; and (c) a candidate peptide;
(ii) growing the cellular host under conditions sufficient to distinguish the expression of each reporter gene(s) at (a) from expression of the reporter gene(s) at (b); and
(iii) detecting those host cells wherein expression of the reporter gene(s) operably under control of the target interaction is(are) partially or completely inhibited and expression of the reporter gene(s) operably under control of the non-target interaction is(are) not inhibited, said detected cells expressing a peptide that partially or completely inhibits the target interaction.

2. The method of claim 1, wherein the binding partners of said target interaction operably and simultaneously control the expression of two distinct reporter genes in said cellular host.

3. The method of claim 1, wherein one or more of the binding partners of the non-target interaction is the same as a binding partner of the target interaction.

4. The method of claim 1, wherein:
(i) the target interaction involves two or more proteinaceous binding partners wherein one of said partners binds to nucleic acid comprising a cis-acting sequence and the other of said partners activates transcription of the reporter gene(s) of the target interaction when the target interaction occurs in the host cell; and
(ii) the non-target interaction involves two or more proteinaceous binding partners wherein one of said partners binds to nucleic acid comprising a cis-acting sequence and the other of said partners activates transcription of the reporter gene(s) of the non-target interaction when the non-target interaction occurs in the host cell.

5. The method of claim 4, wherein:
(i) the proteinaceous binding partners of the target interaction consist of two fusion proteins, wherein (a) one fusion protein comprises the DNA binding domain of a transcription factor and an amino acid sequence that dimerizes with the other fusion protein of the target interaction; and (b) one fusion protein comprises the transcriptional activator domain of a transcription factor and an amino acid sequence that dimerizes with the other fusion protein of the target interaction, and wherein dimerization between said fusion proteins produces a protein complex that binds to the cis-acting sequence and activates expression of the reporter gene(s) when the target interaction occurs in the host cell; and
(ii) the proteinaceous binding partners of the non-target interaction consist of two fusion proteins, wherein (a) one fusion protein comprises the DNA binding domain of a transcription factor and an amino acid sequence that dimerizes with the other fusion protein of the non-target interaction; and (b) one fusion protein comprises the transcriptional activator domain of a transcription factor and an amino acid sequence that dimerizes with the other fusion protein of the non-target interaction, and wherein dimerization between said fusion proteins produces a protein complex that binds to the cis-acting sequence and activates expression of the reporter gene(s) when the non-target interaction occurs in the host cell.

6. The method of claim 5 wherein:
(i) the fusion proteins of the target interaction and the non-target interaction that comprise a transcription activator domain are the same;
(ii) the fusion proteins of the target and non-target interactions that comprise a DNA binding domain are different; and
(iii) the cis-acting sequences of the target and non-target interactions are different.

7. The method of claim 5 wherein:
(i) the fusion proteins of the target and non-target interactions that comprise a DNA binding domain are the same; and
(ii) the cis-acting sequences of the target and non-target interactions are the same; and
(iii) the fusion proteins of the target interaction and the non-target interaction that comprise a transcription activator domain are different.

8. The method of claim 5 wherein:
(i) the fusion proteins of the target and non-target interactions that comprise a transcription activator domain are the same; and
(ii) the cis-acting sequences of the target and non-target interactions are different; and
(iii) the proteins of the target interaction and the non-target interaction that are fused to a DNA binding domain are the same and each of said proteins is fused to a different DNA binding domain.

9. The method of claim 6, wherein the cis-acting sequence of the target interaction or the non-target interaction is selected from the group consisting of: LexA operator, GAL4 binding site, and cl operator and wherein the DNA binding domain of the target interaction or the non-target interaction is selected from the group consisting of: LexA operator binding domain, GAL4 DNA binding domain; and cl operator binding domain.

10. The method of claim 7, wherein the cis-acting sequence of the target interaction and the non-target interaction is selected from the group consisting of: LexA operator, GAL4 binding site, and cl operator and wherein the DNA binding domain of the target interaction and the non-target interaction is selected from the group consisting of: LexA operator binding domain, GAL4 DNA binding domain; and cl operator binding domain.

11. The method according to claim 6 or 7, wherein the transcriptional activator domain is the GAL4 activator domain.

12. The method of claim 6, wherein the fusion proteins of the target interaction and the non-target interaction that comprise a transcription activator domain also comprise a dimerization region of the SCL protein and wherein the fusion proteins of the target and non-target interactions that comprise DNA binding domains each comprise a dimerization region of a distinct protein selected from the group consisting of: LMO1, LMO2, DRG, mSin3A, and E47.

13. The method of claim 12 wherein the fusion protein of the target interaction comprises the dimerization region of LMO2 and wherein the fusion protein of the non-target interaction comprises the dimerization region of E47.

14. The method of claim 12 wherein the fusion protein of the target interaction comprises the dimerization region of LMO2 and wherein the fusion protein of the non-target interaction comprises the dimerization region of mSin3A.

15. The method of claim 12 wherein the fusion protein of the target interaction comprises the dimerization region of E47 and wherein the fusion protein of the non-target interaction comprises the dimerization region of LMO2.

16. The method of claim 12 wherein the fusion protein of the target interaction comprises the dimerization region of E47 and wherein the fusion protein of the non-target interaction comprises the dimerization region of mSin3A.

17. The method of claim 7, wherein the fusion proteins of the target interaction and the non-target interaction that comprise a DNA binding domain also comprise a dimerization region of the SCL protein and wherein the fusion proteins of the target and non-target interactions that comprise transcription activator domains each comprise a dimerization region of a distinct protein selected from the group consisting of: LMO1, LMO2, DRG, mSin3A, and E47.

18. The method of claim 17 wherein the fusion protein of the target interaction comprises the dimerization region of LMO2 and wherein the fusion protein of the non-target interaction comprises the dimerization region of E47.

19. The method of claim 4 wherein the target interaction is between two proteinaceous binding partners that each comprise an RNA-binding domain and a hybrid RNA molecule capable of binding to said RNA-binding domains, wherein one of said proteinaceous binding partners binds to nucleic acid comprising a cis-acting sequence and the other of said proteinaceous binding partners activates transcription of the reporter gene(s) of the target interaction when the target interaction occurs in the host cell.

20. The method according to claim 1 or 2, wherein a reporter gene operably under the control of the target interaction or the non-target interaction encodes a fluorescent protein and wherein said detecting comprises identifying those cells that do not fluoresce or have reduced fluorescence when said reporter gene is not expressed compared to when said reporter gene is expressed.

21. The method of claim 20 wherein the reporter gene is the GFP gene or cobA gene or a variant or fragment of said GFP gene or said cobA gene that encodes a fluorescent protein.

22. The method according to claim 1 or 2, wherein a reporter gene operably under the control of the target interaction or the non-target interaction is a counter selectable reporter gene that encodes a polypeptide capable of converting a non-toxic substrate to a toxic product and wherein said detecting comprises identifying those cells that grow or survive when said counter selectable reporter gene is not expressed.

23. The method of claim 22 wherein the counter selectable reporter gene is selected from the group consisting of: URA3, CYH2, and LYS2.

24. The method of claim 23 wherein two counter selectable reporter genes consisting of URA3 and CYH2 are operably under the control of the target interaction.

25. The method of claim 23 wherein two counter selectable reporter genes consisting of URA3 and LYS2 are operably under the control of the target interaction.

26. The method of claim 23 wherein two counter selectable reporter genes consisting of LYS2 and CYH2 are operably under the control of the target interaction.

27. The method of claim 24 wherein a counter selectable reporter gene operably under control of the non-target interaction is LYS2.

28. The method according to any one of claims 24 to 26 wherein a reporter gene operably under control of the non-target interaction is LEU2.

29. The method of claim 2 wherein multiple reporter genes are operably under the control of the target interaction and wherein said reporter genes comprise at least one counter selectable reporter gene and at least one gene encoding a fluorescent protein such that said detecting comprises identifying those cells grow or survive and do not fluoresce or have reduced fluorescence when said reporter gene is not expressed compared to when said reporter gene is expressed.

30. The method of claim 1 wherein multiple reporter genes are operably under the control of the non-target interaction and wherein said reporter genes comprise at least one counter selectable reporter gene and at least one gene encoding a fluorescent protein.

31. The method of claim 1, wherein the candidate peptide is expressed in a conformationally constrained form within a Trx polypeptide loop or comprising oxidized flanking cysteine residues.

32. The method of claim 1 further comprising selecting and growing the detected cells.

33. The method of claim 1 further comprising the first step of introducing into the cellular host one or more nucleic acids that comprise a sequence selected from the group consisting of:
(i) a sequence encoding a binding partner of the target interaction in an expressible format;
(ii) a sequence encoding a binding partner of the non-target interaction in an expressible format;
(iii) a sequence encoding an activation domain of the target interaction in an expressible format;
(iv) a sequence encoding an activation domain of the non-target interaction in an expressible format;
(v) a sequence encoding a DNA binding domain of the target interaction in an expressible format;
(vi) a sequence encoding a DNA binding domain of the target interaction in an expressible format;
(vii) a sequence encoding the candidate peptide in an expressible format;
(viii) a sequence comprising a cis-acting sequence and a reporter gene in an expressible format; and
(ix) a sequence comprising a cis-acting sequence and a counter selectable reporter gene in an expressible format.

34. The method of claim 33 further comprising mating those cells having one or more of said nucleic acids so as to combine sufficient nucleic acids into a single cell to select those host cells that grow or survive when the counter selectable reporter genes operably under control of the target interaction are expressed.

35. The method of claim 33, wherein nucleic acid encoding a binding partner or a candidate peptide also encodes a nuclear localization signal (NLS) to facilitate nuclear localization of said binding partner or said candidate peptide.

36. The method of claim 33, wherein one or more of said nucleic acids is placed operably under the control of a promoter selected from the group consisting of: MYC, GAL 1, CUP1, PGK1, ADH1, ADH2, PHO4, PHO5, HIS4, HIS5, TEF1, PRB1, GUT1, SPO13, CMV, SV40, LAC, EM7, SV40, and T7.

37. The method of claim 33, wherein one or more of said nucleic acids is contained within a vector selected from the group consisting of: pBLOCK-3.0 (SEQ ID NO: 1); pBLOCK-3.2 (SEQ ID NO: 2); pBLOCK-3.4 (SEQ ID NO: 3); pBLOCK-3.6 (SEQ ID NO: 4); pBLOCK-3.8 (SEQ ID NO: 5); pBLOCK-3.9 (SEQ ID NO: 6); pBLOCK-3.10 (SEQ ID NO: 7); pBLOCK-3.11 (SEQ ID NO: 8); pBLOCK-4.0 (SEQ ID NO: 9); and pRT2 (SEQ ID NO: 10).

38. The method of claim 1 wherein the cellular host is a yeast cell.

39. A method of identifying a peptide that partially or completely inhibits a target interaction between two or more binding partners in a yeast cell but does not inhibit a non-target interaction between some but not all of said binding partners, said method comprising:
(i) transforming a yeast cell with a vector selected from the group consisting of: pBLOCK-3.0 (SEQ ID NO: 1); pBLOCK-3.2 (SEQ ID NO: 2); pBLOCK-3.4 (SEQ ID NO: 3); pBLOCK-3.6 (SEQ ID NO: 4); pBLOCK-3.8 (SEQ ID NO: 5); pBLOCK-3.9 (SEQ ID NO: 6); pBLOCK-3.10 (SEQ ID NO: 7); pBLOCK-3.11 (SEQ ID NO: 8); and pBLOCK-4.0 (SEQ ID NO: 9), wherein said vector further comprises nucleic acid encoding a candidate peptide being tested for inhibitory activity;
(ii) introducing to said transformed yeast cell nucleic acid encoding: (a) the binding partners of said target interaction such that they operably control the expression of one or more counter selectable reporter genes or fluorescent protein-encoding reporter genes in said cellular host, wherein said expression is partially or completely inhibited by disruption of said target interaction; and (b) the binding partners of said non-target interaction such that they operably control the expression of one or more counter selectable reporter genes or fluorescent protein-encoding reporter genes in said cellular host, wherein said expression is partially or completely inhibited by disruption of said non-target interaction and wherein said reporter gene is distinct from the reporter gene(s) expressed under control of the target interaction;
(iii) selecting the recombinants;
(iv) growing the recombinants under conditions sufficient to distinguish the expression of each reporter gene(s) at (a) from expression of the reporter gene(s) at (b); and
(v) detecting those host cells wherein expression of the reporter gene(s) operably under control of the target interaction is(are) partially or completely inhibited and expression of the reporter gene(s) operably under control of the non-target interaction is(are) not inhibited, said detected cells expressing a peptide that partially or completely inhibits the target interaction.

40. The method of claim 39 wherein nucleic acid is introduced at (ii) by means of transformation and the recombinants selected at (iii) are the transformants produced by said transformation.

41. The method of claim 39 wherein nucleic acid is introduced at (ii) by means of cell mating and the recombinants selected at (iii) are diploids arising from said cell mating.

42. The method of claim 39 wherein the fluorescent protein-encoding reporter genes are introduced to the cell by transforming the cell with the vector pRT2 (SEQ ID NO: 11) or by mating the cell with a yeast cell containing said vector.

43. The method of claim 39 wherein nucleic acid encoding one or more of the binding partners of the target interaction and/or the non-target interaction are introduced to the cell by transforming the cell with a derivative of a vector selected from the group consisting of: pBLOCK-3.0 (SEQ ID NO: 1); pBLOCK-3.2 (SEQ ID NO: 2); pBLOCK-3.4 (SEQ ID NO: 3); pBLOCK-3.6 (SEQ ID NO: 4); pBLOCK-3.8 (SEQ ID NO: 5); pBLOCK-3.9 (SEQ ID NO: 6); pBLOCK-3.10 (SEQ ID NO: 7); pBLOCK-3.11 (SEQ ID NO: 8); and pBLOCK-4.0 (SEQ ID NO: 9), wherein said derivative includes a nucleotide sequence encoding said binding partner.

44. The method of claim 43 wherein nucleic acid encoding up to three binding partners of the target interaction and/or the non-target interaction are introduced to the cell by transforming the cell with a derivative of the vector pBLOCK-3.11 (SEQ ID NO: 9), said derivative including nucelotide sequences encoding said binding partners.

45. A peptide that partially or completely inhibits a target interaction between two or more binding partners in a cell but does not inhibit a non-target interaction between some but not all of said binding partners in said cell when detected by the method of claim 1.

46. A peptide that partially or completely inhibits a target interaction between two or more binding partners in a cell but does not inhibit a non-target interaction between some but not all of said binding partners in said cell when detected by the method of claim 39.

47. A yeast shuttle vector comprising a nucleotide sequence substantially as set forth in a sequence selected from the group consisting of SEQ ID NOs: 1 to 10 and a functionally equivalent variant or derivative of any one of said sequence.

48. A vector for expressing red and green fluorescent proteins in yeast comprising:
(i) a green fluorescent protein expression cassette comprising the gfp gene operably under control of a chimeric yeast operable LexA/GAL1 promoter having multiple LexA operator sites; and
(ii) a red fluorescent protein expression cassette comprising the cobA gene operably under control of a chimeric cl/GAL1 promoter having multiple cl operator sites.

49. The vector of claim 48 having the structural characteristics for expression of the fluorescent proteins as contained in vector pRT2 (SEQ ID NO: 11).

50. A method for determining the effect of a peptide on a eukaryotic cell comprising:
(v) isolating a nucleotide sequence encoding a peptide inhibitor identified by the method of claim 1;
(vi) transfecting said eukaryotic cell with the isolated nucleic acid; and
(vii) comparing the phenotype or expression pattern of the transfected eukaryotic cell to the phenotype or expression pattern of an otherwise isogenic non-transfected cell, wherein a different phenotype or expression pattern indicates that the peptide has an effect on the cell.

51. The method of claim 50 wherein the eukaryotic cell is a mammalian cell.

52. The method of claim 50 wherein comparing the expression pattern of the transfected eukaryotic cell to the expression pattern of an otherwise isogenic non-transfected cell is performed by producing an array of protein or nucleic acid expressed by the transfected and non-transfected cells and comparing said protein or nucleic acid.

53. The method of claim 50 wherein the phenotype of the transfected cell is compared to the phenotype of the non-transfected cell.

54. A process for identifying a peptide for the prophylactic or therapeutic treatment of a mammal comprising performing the method according to any one of claims 50 to 53 on a diseased cell and selecting a peptide that reverts the phenotype or expression profile of the transfected cell.

55. A process for identifying a binding partner or drug target comprising performing the method of claim 52 and identifying the nucleic acid or protein that is modified in the transfected cell.
